(19) United States
(12) Patent Application Publication
(10) Pub. No.: US 2010/0010101 A1
(43) Pub. Date: Jan. 14, 2010

(54) Rapid-Melt Compositions and Methods of Making Same

(75) Inventor: S. Rao Cherukuri, Vienna, VA (US)

Correspondence Address: BLANK ROME LLP, ONE LOGAN SQUARE, PHILADELPHIA, PA, 19103, US

(73) Assignee: Capricorn Pharma, Inc., Frederick, MD (US)

(21) Appl. No.: 12/501,652

(22) Filed: Jul. 13, 2009

Related U.S. Application Data
(63) Continuation-in-part of application No. 11/818,212, filed on Jun. 12, 2007.
Application No. 11/818,212 is a continuation of application No. 10/208,877, filed on Aug. 1, 2002, now Pat. No. 7,229,641.
Application No. 10/208,877 is a continuation-in-part of application No. 09/898,471, filed on Jul. 5, 2001, now Pat. No. 6,406,717.
Application No. 09/898,471 is a continuation-in-part of application No. 09/858,885, filed on May 17, 2001, now Pat. No. 6,589,556.
Application No. 09/858,885 is a continuation-in-part of application No. 09/610,489, filed on Jul. 5, 2000, now Pat. No. 6,375,982.

(51) Int. Cl. A61K 47/04; A61K 47/12

(52) U.S. Cl. 514/770; 514/784

(57) ABSTRACT

The present invention provides a novel rapid-melt pharmaceutical composition comprising a binder, a super emulsifier, a diluent/bulking material; and an active ingredient. The present invention also provides a rapid-melt bead composition comprising a binder having a melting point from about 20 to about 90° C., an emulsifier; a diluent/bulking material; and an active ingredient. Further, the present invention provides a chew tablet composition comprising a binder; an emulsifier having a melting point of greater than about 40° C.; a diluent/bulking material; and an active ingredient. Methods of making the disclosed compositions are also described.

### PRIORITY CLAIM

This application is a continuation-in-part (CIP) of U.S. patent application Ser. No. 11/818,212, filed Jun. 12, 2007, which is a continuation-in-part of U.S. patent application Ser. No. 10/208,877, filed Aug. 1, 2002, which is a continuation-in-part of U.S. patent application Ser. No. 09/898,471, filed Jul. 5, 2001, now issued as U.S. Pat. No. 6,406,717, which is a continuation-in-part of U.S. patent application Ser. No. 09/858,885, filed May 17, 2001, now issued as U.S. Pat. No. 6,589,556, which is a continuation-in-part of U.S. patent application Ser. No. 09/610,489, filed Jul. 5, 2000, now issued as U.S. Pat. No. 6,375,982, each of which is incorporated herein by reference.

### FIELD OF THE INVENTION

The present invention relates to a rapid-melt composition for delivery of prophylactic and therapeutic active materials to a mammal, methods of making the same, and methods of using the same. Preferably, the prophylactic or therapeutic active is a psychotropic, a gastrointestinal therapeutic or a migraine therapeutic.

### DESCRIPTION OF THE PRIOR ART

Pharmaceutical compositions may be produced in a variety of dosage forms, depending upon the desired route of administration of the therapeutic material. Oral dosage forms, for example, include such solid compositions as tablets, beads/granules, emulsions, and suspensions. The particular dosage form utilized will depend on such factors as the solubility and chemical reactivity of the pharmaceutical active. Further, the dosage form may be selected so as to optimize delivery of the pharmaceutical active and/or consumer acceptability of the composition.

Tablet oral dosage forms compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active. However, liquid formulations may offer advantages in the treatment of certain disorders, such as disorders of the upper gastrointestinal tract, wherein delivery of an active material dissolved or dispersed in a liquid ensures rapid and complete delivery to the afflicted area. In an effort to obtain the therapeutic advantages associated with liquid formulations as well as the broad advantages associated with solids, many chewable tablet formulations have been developed.

One important factor in formulating oral dosage forms such as chewable tablets, beads, powders, granules, is palatability and mouth feel, especially in tablets that include pharmaceutical dosages. Many pharmaceutical and confectionery tablets are designed to be chewed either to provide proper flavor or to increase the surface area of a particular drug to permit rapid activity in the digestive tract or circulatory systems. However, many pharmaceutical ingredients usually have both an unpleasant mouth feel and unpalatable taste due to chalkiness, grittiness, dryness and astringent properties of these materials. Accordingly, the practical value of these materials is substantially diminished since patients finding them objectionable may fail to take them as prescribed. A number of formulations have been investigated to ease the mouth feel and palatability of such compositions.

Khankari et al., U.S. Pat. No. 6,024,981, discloses a rapidly dissolving robust dosage form directed to a hard tablet that can be packaged, stored and processed in bulk. The solid tablet dissolves in the mouth of a patient with a minimum of grit. The tablet contains an active ingredient mixed into a matrix of a non-direct compression filler and a relatively high lubricant content.

Amselem, U.S. Pat. No. 5,989,583, discloses a dry solid lipid composition suitable as an oral dosage form. The composition contains a lipophilic substance, at least one fat which is a solid at about 25° C. and at least one phospholipid present in an amount of about 2 to 40% by weight of the composition. However, the resultant product is a dry solid lipid composition.

United Kingdom patent application GB 2 195 892 discloses pharmaceutical chewable tablets with improved palatability. The lipid-containing tablets include a lipid material having a melting point from about 26° C. to about 37° C., a particulate dispersant material, an emulsifier and a safe and effective amount of a pharmaceutically active material. The tablets of the lipid composition exhibit improved palatability, and effective dispersion in the mouth and stomach.

United Kingdom patent application GB 2 195 891 also discloses pharmaceutical chewable tablets with improved palatability. The lipid-containing tablets include a lipid material, a dispersant, a nonionic emulsifier having an HLB of at least 10, and a safe and effective amount of a pharmaceutical active material, wherein the average HLB of all emulsifiers in the composition is at least about 8.

Nakamichi et al., U.S. Pat. No. 5,837,285, discloses fast soluble tablets that can be produced by a simple method. The tablet base is a sugar alcohol. The mixture of the sugar alcohol and a drug is subjected to compressive shaping prior to drying in the process. The dry solid tablet can be produced by modification of conventional tableting technology and possesses physico-chemical stability.

Chavkin et al., U.S. Pat. No. 5,753,255 discloses a chewable medicinal tablet. The tablet contains about 30 to about 95% by weight of a capric triglyceride and a medicinally active ingredient up to 60% by weight. If the medicinally active ingredient is less than about 30% by weight, then the composition also contains up to 10% by weight of a member of the group consisting of glyceryl monostearate, a mixture of glyceryl monostearate and glyceryl monopalmitate, and a mixture of glyceryl monostearate and glyceryl distearate.

Geyer et al., U.S. Pat. No. 5,320,848, discloses a nonaqueous chewable composition for oral delivery of unpalatable drugs. The drug is intimately dispersed or dissolved in a pharmaceutically-acceptable lipid that is solid at room temperatures. The lipid material desirably readily melts with the application of mild temperatures, i.e. about 55 to 95 C.

Lapidus, U.S. Pat. No. 4,937,076, discloses a chewable aspirin and buffering material tablet in a single dosage form. The buffering materials are integrally dispersed and bound in a fatty material of chocolate, synthetic chocolate or hydrogenated tallow. The fatty material individually coats the aspirin and buffering material.

Valentine, U.S. Pat. No. 4,684,534, discloses quick-liquefying, chewable tablets. The tablets have a harder outer shell which inhibits penetration of liquid, and a softer interior which quickly liquefies when the tablet and shell are broken into pieces and contacted by the liquid. The excipient or base material of the tablet is made from carbohydrates held together with small quantities of a carbohydrate binder such as maltodextrin. The tablets can contain active ingredients such as pharmaceuticals, breath sweeteners, vitamins and dietary supplements.

Morris et al., U.S. Pat. No. 4,609,543, discloses a soft homogeneous antacid tablet. The tablet contains solid antacid particles thoroughly coated with a mixture composed of a fatty material or oil, a surfactant, and a flavor. The fat or oil is present in an amount of from about 25% to about 45% of the mixture. The primary particle size of the antacid is less than 100 millimicrons.

Fountaine, U.S. Pat. No. 4,446,135, discloses chewable calcium carbonate-containing antacid tablets having good mouth feel properties. The good mouth feel properties of the tablet are obtained by using calcium carbonate of a particular particle size in combination with certain excipients. The calcium carbonate is present in an effective amount and has a size from about 5 to 50 microns in diameter.

Puglia et al., U.S. Pat. No. 4,327,077, discloses a compressed chewable antacid tablet which has good flexibility, is breakage resistant and disintegrates immediately upon chewing. The tablet is formed of a recrystallized fatty material, such as chocolate, a bulking material and an active ingredient bound up in the particles of the recrystallized fatty material. The preferred recrystallized fatty material is a chocolate or a synthetic chocolate.

Puglia et al., U.S. Pat. No. 4,327,076, also discloses a compressed chewable antacid tablet which has good flexibility, is breakage resistant and disintegrates immediately upon chewing. The tablet is formed of particles of the antacid or other active ingredients which are admixed with particles formed of edible fat or oil absorbed on a fat-absorbing material, such as microcrystalline cellulose. Upon chewing, the tablet is quickly converted to a smooth creamy non-gritty palatable emulsion.

However, the prior art compositions contain various disadvantages. For example, tablets may be incompletely chewed due to the poor palatability of the composition. Such compositions may also have a gummy texture, and are subject to “taste fatigue,” i.e., the composition is perceived to be less palatable after ingestion of multiple doses. Further, the binders and other materials used in such chewable tablets may prevent rapid and effective delivery of active materials to the stomach.

There is a need for a rapid-melt, composition that behaves like a liquid when consumed by a mammal, and yet acts like a solid in many other ways. The need extends for compositions in which little to substantially no biting or chewing is necessary in order for the composition to melt, disintegrate, decompose, or otherwise break down or apart in the mouth of a mammal. Such compositions are ideal for uses in the fields of pediatric and geriatric care, that is, for use with people or mammals that do not have any teeth. These compositions are particularly useful for pediatric, geriatric patients or for those with limited ability to swallow traditional dosage forms.

It has been found that product formulations containing one or more certain lipid materials, emulsifiers and particulate materials are highly palatable and effective compositions for the delivery of pharmaceutical active materials. Such compositions afford better taste, mouth feel and storage stability than those compositions known in the art

### BRIEF SUMMARY OF THE INVENTION

Applicant has unexpectedly developed a method of preparing a rapid-melt composition comprising the steps of:

a) melting at least one binder in an amount from about 0.01% to about 70% by weight with a salivating agent in an amount from about 0.05% to about 15% by weight, to form a first mixture;

b) mixing a therapeutically effective amount of an active ingredient with at least one lubricant to form a second mixture;

c) combining said first mixture with said second mixture to form a compressible mixture; and

d) compressing said compressible mixture into said rapid-melt composition.

Applicant has further developed a method of preparing a rapid-melt composition comprising the steps of:

a) melting at least one binder in an amount from about 0.01% to about 70% by weight with a salivating agent in an amount from about 0.05% to about 15% by weight, to form a first mixture;

b) mixing a therapeutically effective amount of an active ingredient with at least one lubricant to form a second mixture;

c) combining said first mixture with said second mixture to form a compressible mixture;

d) compressing said compressible mixture into said rapid-melt composition;

e) heating said rapid-melt composition to a temperature 40 to 60° C. for a period of 1 to 10 minutes in order to convert said binder to a bonding agent; and

f) cooling said heated rapid-melt composition.

Further, Applicant has unexpected developed a method for preparing a compressed rapid-melt composition comprising the steps of:

a) mixing at least one diluent present in an amount of 0.1 to 99%, which is good for low dose drugs, by weight with a therapeutically effective amount of an active ingredient and a binding agent in an amount which is less than required to fully bind said diluent and said active ingredient;

b) granulating said mixture from step a) to form granules;

c) mixing said granules with a bonding agent in an amount of 0.01 to 30% by weight to form a compressible mixture; and

d) compressing said compressible mixture into said rapid-melt composition.

The rapid-melt molded compositions of the present invention contains a binder a salivating agent, a diluent/bulking material, and an active ingredient. The compositions exhibit good resistance to prolonged exposure to heat and the atmosphere. More particularly, the compositions surprisingly maintain their texture and rapid melting properties when exposed to those elements.

### DETAILED DESCRIPTION OF THE EMBODIMENTS

The rapid-melt compositions of the present inventive subject matter contains at least one binder, a salivating agent, an active material, and a diluent/bulking material. The rapid-melt compositions may also contain a slipping agent to aid in the transport of the composition from the mouth of the mammal to the stomach thereof.

As used herein, the expression “mammal” includes without limitation any mammalian subject, such as mice, rats, guinea pigs, cats, dogs, human beings, cows, horses, sheep or other livestock.

As used herein, the expression “free water” means water that is not found in other ingredients. Many ingredients used in the present inventive compositions may also have water as part of the ingredient, and the term “free water” refers to water that is separate from those ingredients.

The unique novel combination of elements allows for fast melting of the composition when placed in the mouth of a user. By pressing the composition between the tongue and cheek of the user, the saliva of the user provides hydration to the composition and allows the composition to melt without any chewing. A unique feature of the present inventive compositions is that the composition becomes a liquid upon the application of pressure. The compositions rapidly melt upon the application of pressure by the tongue of the patient, thus forming a liquid carrier for the active ingredients contained therein. The liquid helps provide the unique characteristics and features of the present inventive compositions.

The liquification of the inventive compositions can be achieved through the application of pressure by the tongue of the patient, as described above. Optionally, the liquification may be attained by the patient chewing the compositions. A slight amount of chewing will enhance the liquefication of the compositions. A further way for the composition to be liquefied is by the patient sucking on the rapid-melt, compositions of the inventive subject matter.

The rapid-melt technology of the present inventive subject matter has multiple applications which are ideal for the unique properties of the compositions. One such application is the delivery of active ingredients to a mammal in need thereof.

In addition, the melting feature of the novel compositions makes the compositions ideal for uses in pediatric and geriatric care, since small children and aged individuals often have difficulty chewing items. With this intended use in mind, the compositions may be specially formulated for pediatric and geriatric patients. The unique properties will aid in drug compliance by such patients as the drugs may be administered in a way that will not require chewing by the patient.

Another application for which the inventive compositions are ideal is to enhance the saliva flow of a patient. A frequent problem for geriatric patients is dry-mouth, or the inability to salivate sufficiently. The aid of saliva flow by the use of the present inventive compositions will enhance tooth cleaning within the patient, as well as stimulate better drug delivery to the patient. Also, the increased saliva flow will facilitate better breath characteristics in the patient. The use of xylitol, as well as other polyols and sugars, food acids, and binder-emulsifiers in the inventive compositions will contribute to the enhancement of the saliva flow of the patient.

A further application for the inventive compositions would be the preparation of compositions for drug delivery in diabetic patients. A diabetic patient must monitor the intake of sugar and the ability to formulate the present inventive compositions with maltitol and other non-cariogenic components makes them ideal for delivery of drugs to diabetic patients.

The rapid-melt compositions of the present inventive subject matter are preferably anhydrous, that is, they do not contain any water. The lack of water in the inventive compositions allows high doses of active materials or combinations of active materials to be incorporated into the compositions due to the stability of the active materials in the absence of the water. It is contemplated, however, that the compositions may optionally include an amount of water. The amount of water present will depend on the active ingredients to be delivered, but generally will be present in an amount less than 2.0% by weight of the composition. Preferably, the water will be present in an amount less than 1.0% by weight of the composition.

The rapid-melt compositions of the present inventive subject matter contain at least one binder. As used herein, “binder” means at least one ingredient useful in keeping the composition in its state, may be either solid or liquid, and may include, without limitation, a high melting point fat or waxy material such as lipid materials, polyethylene glycols (PEG), waxes and other fats. Preferably, the compositions of the present inventive subject matter contains a mixture of binders. The solid binders useful in the compositions of the present inventive subject matter have a melting point of about 25 to 90° C., and preferably about 37° C. When more than one binder is used in the inventive compositions, the melting point of the combination of the binders will remain within the range of 25 to 90° C., and preferably about 37° C. The inventive subject matter contemplates the use of mixtures of solid binders and liquid binders. For a non-limiting example, the present inventive subject matter contemplates mixing a small amount of a high-melting point lipid with a liquid binder to achieve a binder that attains the desired product characteristics. These characteristics include such factors as mouth feel, rapidity of melting in the mouth, appearance, flavor and compatibility with active materials and therapeutic active materials.

Among the lipid materials useful as binders in the compositions of the present inventive subject matter are those which are commercially available and commonly used in confectionery and other food products. Such lipid materials include, without limitation, cocoa butter, hydrogenated tallow, hydrogenated vegetable oils, hydrogenated cotton seed oil, palm kernel oil, soybean oil, stannol esters, and derivatives and mixtures thereof. Hydrogenated vegetable oils (such as hydrogenated palm kernel oil), cocoa butter, and cocoa butter substitutes are among the preferred useful lipid materials. Additional binders may include emulsifiers, surface active agents, plasticizers, such as glycerol esters, polyalcohol esters, polyoxyethylene esters of hydrophilic and hydrophobic balances from 0.5 to above 20 and polyethylene glycols. Other examples include saccharides such as monosaccharides and oligosaccharides. Examples of monosaccahrides include: dextrose, dextrose monohydrate, lactose, mannose, fructose, etc. Liquid binders may also be used. Examples of liquid binders are, without limitation, polysaccharides, gum solutions, water, corn syrup, hydrogenated starch hydrolysates, glycerine, polypropylene glycol, polyethylene glycols, and mixtures thereof. It should be noted that liquid binders, when used may be present in quantities to not affect the constituency of the product so that the final product retains a predominantly solid constituency. In some aspects, the liquid binders may not exceed about 5% of the composition.

The amount of binder present in the rapid-melt composition of the present inventive subject matter is from about 0.01% to about 70% by weight of the final composition. Preferably, the amount of binder is from about 0.01% to about 50% by weight of the composition. More preferably the binder is present from about 5% to about 30% by weight of the composition.

The binder is used to provide good melt away properties to the composition while preventing a gritty texture being imparted by the composition. The binder aids in the fast melting of the composition when placed in the mouth of a user.

The rapid-melt composition of the present inventive subject matter also contains a salivating agent. As is used herein, “salivating agent” means a material that promotes greater salivation in the user of the compositions of the present inventive subject matter. The salivating agent helps create salivation in the mouth of the mammal using the inventive compositions. This is an important feature since the present compositions are intended to be taken by the patient without the aid of water to help in the transporting of the composition to the stomach of the patient. The salivating agent can be, without limitation, an emulsifier or a food acid that initiates salivation in the mouth of the patient.

Examples of emulsifiers useful as salivating agents in the compositions of the present inventive subject matter include, without limitation, alkyl aryl sulfonates, alkyl sulfates, sulfonated amides and amines, sulfated and sulfonated esters and ethers, alkyl sulfonates, polyethoxlyated esters, mono-, di-, and triglycerides, diacetyl tartaric esters of monoglycerides, polyglycerol esters, sorbitan esters and ethoxylates, lactylated esters, phospholipids such as lecithin, polyoxyethylene sorbitan esters, proplyene glycol esters, sucrose esters, and mixtures thereof. The emulsifier may be either saturated or unsaturated. It should be noted that some of the emulsifiers that are salivating agents may also function as binders.

In the case where the active ingredient is present at high concentration, such as greater than 4%, preferably greater than 40%, most preferably greater than 70%, the preferred binder used are glycerides, lecithin, or a combination thereof. Also, in this case, the emulsifier are also preferably used as binders. The combination of glycerides and lecithin, as emulsifier and binder, unexpectedly allows for a chew tablets having high concentrations of active ingredient(s).

In certain embodiments, the emulsifier used are referred to herein as a “super emulsifier.” The term “super emulsifier,” as used herein, refers to an emulsifier having a hydrophilic-lipophilic balance (HLB) of greater than about 10, preferably greater than about 14. A HLB value ranges from 0 to 20, where a low value corresponds to a hydrophobic molecule and a high value corresponds to a hydrophilic molecule. Several methods can be used to determine HLB. The method preferred by the present invention is Griffin's method (HLB value of 0 corresponds to a completely hydrophobic molecule, and a value of 20 would correspond to a molecule made up completely of hydrophilic components). Patel et al., U.S. Pat. No. 6,248,363, which is incorporated herein by reference, disclose a variety of surfactants having HLB greater than 10 that can appropriately be used as super emulsifiers for the present invention. The super emulsifiers include, but are not limited to, those listed in a) to p) as follows:
a) Polyethoxylated fatty acids, e.g., polyethylene glycol (PEG) 400 distearate; PEG 100, 200, 300 monolaurate; PEG 100, 200, 300 monooleate; PEG 400 dioleate; PEG 400-1000 monostearate; PEG-7 oleate; PEG-6 laurate; PEG-7 laurate; PEG-8 laurate; PEG-8 oleate; PEG-8 stearate; PEG-9 oleate; PEG-9 stearate; PEG-10 oleate; PEG-10 stearate; PEG-12 oleate; PEG-12; PEG-12 stearate; PEG-15 oleate; PEG-20 laurate; PEG-20 oleate; PEG-25 stearate; PEG-32 laurate; PEG-32 stearate; PEG-30 stearate; PEG-40 oleate; PEG-40 stearate; PEG-45 stearate; PEG-55 stearate; PEG-100 oleate; PEG-100 stearate; PEG-200 oleate; PEG-400 oleate; and PEG-600 oleate.
b) PEG-fatty acid diesters, e.g., PEG-8 dilaurate; PEG-8 distearate; PEG-10 dipalmitate; PEG-12 dilaurate; PEG-12 distearate; PEG-12 dioleate; PEG-20 dilaurate; PEG-20 dioleate; PEG-20 distearate; PEG-32 dilaurate; PEG-32 dioleate; PEG-32 distearate; PEG-400 dioleate; and PEG-400 distearate.
c) PEG glycerol fatty acid esters, e.g., PEG-20 glyceryl laurate; PEG-30 glyceryl laurate; PEG-15; PEG-40 glyceryl laurate; PEG-20 glyceryl stearate; PEG-20 glyceryl oleate; and PEG-30 glyceryl oleate.
d) Transesterification products of oils and alcohols, e.g., PEG-20 castor oil; PEG-23 castor oil; PEG-30 castor oil; PEG-40 castor oil; PEG-56 castor oil; PEG-60 castor oil; PEG-100 castor oil; PEG-200 castor oil; PEG-20 hydrogenated castor oil; PEG-25 hydrogenated castor oil; PEG-30 hydrogenated castor oil; PEG-40 hydrogenated castor oil; PEG-45 hydrogenated castor oil; PEG-60 hydrogenated castor oil; PEG-80 hydrogenated castor oil; PEG-100 hydrogenated castor; PEG-20 almond glycerides PEG-25 trioleate; PEG-40 palm kernel oil; PEG-60 corn glycerides; PEG-60 almond glycerides; PEG-8 caprylic/capric glycerides; PEG-6 caprylic/capric glycerides; lauroyl macrogol-32 glyceride; and stearoyl macrogol glyceride.
e) Polyglycerized fatty acids, e.g., polyglyceryl-10 laurate; polyglyceryl-10 stearate; and polyglyceryl-10 linoleate.
f) Sterol and sterol derivatives, e.g., PEG-24 cholesterol ether; PEG-30 chlestanol; PEG-25 phytosterol; and PEG-30 soya sterol.
g) PEG sorbitan fatty acid esters, e.g., PEG-10 sorbitan laurate; PEG-20 sorbitan monolaurate; PEG-4 sorbitan monolaurate; PEG-80 sorbitan monolaurate; PEG-6 sorbitan monolaurate; PEG-20 sorbitan monopalmitate; PEG-20 sorbitan monostearate; PEG-8 sorbitan monostearate; PEG-6 sorbitan monostearate; PEG-20 sorbitan tristearate; PEG-60 sorbitan tetrastearate; PEG-20 sorbitan monooleate; PEG-40 sorbitan oleate; PEG-20 sorbitan trioleate; PEG-30 sorbitan tetraoleate; PEG-40 sorbitan tetraoleate; and PEG-20 sorbitan.
h) PEG alkyl ethers, e.g., PEG-23 lauryl ether, laureth-23; PEG-10 cetyl ether; PEG-20 cetyl ether; PEG-10 stearyl ether; PEG-20 stearyl ether; and PEG-100 stearyl ether.
i) Sugar esters, e.g., sucrose distearate/monostearate; sucrose monostearate; sucrose monopalmitate; and sucrose monolaurate.
j) PEG alkyl phenols, e.g., PEG-10-100 nonyl phenol and PEG-15-100 octyl phenol ether.
k) Polyoxyethylene (POE)-polyoxypropylene (POP) copolymers and block copolymers (poloxamers), where the hydrophilic POE and lipolipic POP moieties are in ratios that provide a HLB of greater than about 10.
l) Ionic surfactants; which may include fatty acid salts, e.g., Sodium caproate, Sodium caprylate, Sodium caprate, Sodium laurate, Sodium mynstate, Sodium myristolate, Sodium palmitate, Sodium palmitoleate, Sodium oleate, Sodium ricinoleate, Sodium linoleate, Sodium linolenate, Sodium stearate, Sodium lauryl sulfate (dodecyl), Sodium tetradecyl, sulfate Sodium lauryl sarcosinate, Sodium dioctyl sulfosuccinate [sodium docusate (Cytec)], bile salts, e.g., Sodium cholate, Sodium taurocholate, Sodium glycocholate, Sodium deoxycholate, Sodium taurodeoxycholate, Sodium glycodeoxycholate, Sodium ursodeoxycholate, Sodium chenodeoxycholate, Sodium taurochenodeoxycholate, Sodium glyco cheno deoxycholate, Sodium cholylsarcosinate, Sodium N-methyl taurocholate, Sodium lithocholate, phospholipids, e.g., Egg/Soy lecithin [Epikuron™ (Lucas Meyer), Ovothin™ (Lucas Meyer)], Lyso egg/soy lecithin, Hydroxylated lecithin, Lysophosphatidylcholine, Cardiolipin, Sphingomyelin, Phosphatidyichoiine, Phosphatidyl effianolamine, Phosphatidic acid, Phosphatidyl glycerol, Phosphatidyl serine, phosphoric acid esters, e.g., Diethanolammonium polyoxyethylene-10 oleyl ether phosphate, Esterification products of fatty alcohols or fatty alcohol, ethoxylates with phosphoric acid or anhydride, carboxylates, e.g., Ether carboxylates (by oxidation of terminal OH group of fatty alcohol ethoxylates), Succinylated monoglycerides [LAMEGIN ZE (Henkel)], Sodium stearyl filmarate, Stearoyl propylene glycol hydrogen succinate, Mono/diacetylated tartaric acid esters of mono- and diglycerides, Citric acid esters of mono- and diglycerides, Glyceryl-lacto esters of fatty acids (CFR ref 172.352), Acyl lactylates, e.g., lactylic esters of fatty acids, calcium/sodium stearoyl-2-lactylate, calcium/sodium stearoyl lactylate, Alginate salts, Propylene glycol alginate, sulfates and sulfonates, e.g., Ethoxylated alkyl sulfates, Alkyl benzene sulfones, α-olefin sulfonates, Acyl isethionates, Acyl taurates, Alkyl glyceryl ether sulfonates, Octyl sulfosuccinate disodium, Disodium undecylenamideo-MEA-sulfosuccinate, cationic surfactants, e.g., Lauroyl carnitine, Palmitoyl carnitine, Myristoyl carnitine, Hexadecyl triammonium bromide, Decyl trimethyl ammonium bromide, Cetyl trimethyl ammonium bromide, Dodecyl ammonium chloride, Alkyl benzyldimethylammonium salts, Diisobutyl phenoxyethoxydimethyl benzylammonium salts, Alkylpyridinium salts, Betaines (trialkylglycine), Lauryl betaine (N-lauryl,N,N-dimethylglycine), Ethoxylated amines, Polyoxyethylene-15 coconut amine.
m) Polyoxyethylene fatty acid esters, e.g., polyoxyethylene stearic acid esters of the type available under the trade name MYRJ, e.g., MYRJ 52 (a polyoxyethylene 40 stearate). Other related products include polyethoxylated saturated hydroxy fatty acids which may be produced by reacting a saturated hydroxy fatty acid, e.g., C.sub.18 to C.sub.20 fatty acid, with ethylene oxide or polyethylene glycol. Suitable examples for the present invention include those known in the art and commercially available, e.g., from the BASF company under the trade mark SOLUTOL. Especially preferred is SOLUTOL HS 15 which is known, e.g., from the BASF technical leaflet MEF 151E (1986), to comprise of about 70% polyethoxylated 12-hydroxystearate by weight and about 30% by weight unesterified polyethylene glycol component.
n) Polyoxyetheylene-sorbitan fatty acid esters (polysorbates), e.g., the mono- and trilauryl, palmityl, stearyl and oleyl esters, for instance the polyoxyethylene sorbitan monooleates available under the trade name of TWEEN, such as TWEEN 20, 21, 40, 60, 61, 65, 80, 81 and 85, of which class TWEEN 80 (polysorbat 80) is especially preferred.
o) Reaction products of a natural or hydrogenated castor oil and ethylene oxide. The natural or hydrogenated castor oil may be reacted with ethylene oxide in a molar ratio of from about 1:35 to about 1:60, with optional removal of the polyethyleneglycol component from the products. Various such surfactants are commercially available. Suitable surfactants include polyethyleneglycol-hydrogenated castor oils available under the trade name CREMOPHOR, e.g., CREMOPHOR RH 40 (polyoxyl 40 hydrogenated castor oil) and CREMOPHOR EL (polyoxyl 35 castor oil).
p) Polyoxyethylene glycol long-chain alkyl ethers, such as polyoxyethylated glycol lauryl ether.
   In a preferred embodiment, the super emulsifier is sodium lauryl sulfate, polysorbate 80, or a combination thereof.

Examples of food acids useful as salivating agents in the inventive compositions include, without limitation, citric acid, malic acid, tartarate, food salts such as sodium chloride and salt substitutes, potassium chloride, and mixtures thereof.

The amount of salivating agent present in the rapid-melt composition of the present inventive subject matter is from about 0.05% to about 15% by weight of the final composition. Preferably, the amount of salivating agent from about 0.3% to 0.4% by weight of the composition.

Keeping the amount of salivating agent present in the inventive composition within these limits for weight percentage is important to enhance the desirable properties of the compositions. More particularly, the low amount of salivating agent present in the compositions aid in the compositions retaining the physical state and the rapidity of melting in the mouth of a mammal.

The rapid-melt compositions of the present inventive subject matter further contain a diluent/bulking material. The use of a diluent/bulking material is necessary to serve as a free-flow imparting agent which aids in the moisturizing of the composition when chewed, that is, the diluent/bulking material aids in the processability of the compositions. The diluent/bulking material also serves to reduce the concentration of the active materials and add bulk to the composition. Examples of diluent/bulking materials useful in the compositions of the present inventive subject matter include, without limitation, silicon dioxide, sugars, starches, lactose, sucrose, sorbitol, fructose, talc, stearic acid, magnesium stearate, dicalcium phosphate, erythitol, xylitol, mannitol, maltitol, isomalt, dextrose, maltose, lactose, microcrystalline celluloses and mixtures thereof. It should be noted that some of the diluents/bulking materials also function as binders.

The amount of diluent/bulking material present in the rapid-melt compositions is from about 0.5% to about 99% by weight of the final composition. Preferably, the amount of diluent/bulking material is from about 2% to about 95% by weight of the final composition.

The rapid-melt compositions of the present inventive subject matter may optionally contain a further slipping agent to aid in the palatability of the composition after it melts in the mouth of the mammal. The slipping agent may be a further lipid material, as is described above for binders, or another material which aids in the “slipping” of the composition through the mouth and down the esophagus of the mammal.

The compositions of the present invention may further include a disintegrant, which aid in the break up of the compacted composition when it is put into a fluid environment. Disintegrants can be added to the present invention compositions to promote the breakup of the tablet into smaller fragments in an aqueous environment thereby increasing the available surface area and promoting a more rapid release of the active drug substance. The mechanism for the desirable disintegrant action is usually any one or combination of the disintegrant's ability to swell in an aqueous environment, enhance porosity and provide these pathways into the tablet, (i.e., liquid is drawn up or “wicked” into these pathways through capillary action and rupture the interparticulate bonds causing the tablet to break apart), and change shape in water (i.e., disintegrant molecules are deformed during the tableting process by the compression force, addition of water facilitates the disintegrant molecules to overcome the adhesiveness of the other ingredients of the tablet and return to a more relaxed form. A disintegrant can be added to a powder blend for direct compression or encapsulation. It can also be used with products that are wet granulated. Some tablet fillers can aid in disintegration, examples of which are starch, pregelatinized starch (Starch 1500), and microcrystalline cellulose.

More effective agents referred to as superdisintegrants. These superdisintegrants are more effective in lower concentrations than starch, and has less effect on compressibility and flow ability. Superdisintegrants swell to many times their original size when placed in water while producing minimal viscosity effects. Major groups of superdisintegrants are modified starches, cross-linked polyvinylpyrrolidone, and modified cellulose. Modified starches provide rapid and extensive swelling with minimal gelling. Effective concentration is generally between 0.5-10%, preferably between 1-6%. An examples of modified starches is sodium carboxymethyl ctarch (chemically treated potato starch, i.e. sodium starch glycolate (Explotab, Primogel)). Cross-linked polyvinylpyrrolidone is water insoluble and strongly hydrophilic. It provides strong water wicking, and swelling. The effective concentration is generally between 0.5-5%, and preferably between 1-4%. An example of cross-linked polyvinylpyrrolidone is crospovidone (Polyplasdone XL, Kollidon CL). Modified cellulose is an internally cross-linked form of sodium carboxymethyl cellulose, i.e., croscarmellose sodium. Modified cellulose provides wicking due to fibrous structure, and swelling with minimal gelling. Effective concentration is generally between: 0.5-5%. Examples of modified cellulose are Ac-Di-Sol (Accelerates Dissolution), Nymcel. Another example of superdisintegrant is low-substituted hydroxypropyl cellulose (L-HPC. L-HPC is a low-substituted hydroxypropyl ether of cellulose within quite a small portion of the hydroxypropyl groups in the glucose unit. The typical molar substitution is about 0.2-0.4. It is insoluble in water, and provides rapid swelling in water. Some superdisintegrants can also be used as binders.

The compositions of the present invention may be compressed into tablets or made into granules, beads or particles for direct consumption/administration. The granules, beads or particles may be further processed into additional dosage forms such as tablets, capsules, caplets or suspensions and emulsions.

As discussed above, the preferably anhydrous nature of the present inventive compositions allows for very high doses of active materials to be incorporated therein. The amount of active material present in the inventive compositions will vary depending on the particular active used, but generally will be present in an amount of about 0.001% to 70% by weight of the composition. Preferably, the active ingredients used in the inventive compositions are prophylactic or therapeutic active ingredients. Prophylactic or therapeutic active materials which can be used in the present invention are varied. A non-limiting list of such materials includes the following: antitussives, antihistamines, decongestants, alkaloids, mineral supplements, laxatives, vitamins, antacids, ion exchange resins, anti-cholesterolemics, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, antidrrheal preparations, anti-anginal drugs, vasodialators, anti-hypertensive drugs, vasoconstrictors, migraine treatments, antibiotics, tranquilizers, anti-psychotics, antitumor drugs, anticoagulants, antithrombotic drugs, hypontics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiashmatics, cough suppressants, mucolytics, anti-uricemic drugs and mixtures thereof.

Preferred prophylactic or therapeutic active materials contemplated for use in the present inventive subject matter are analgesics. Examples of pain medication such as analgesics useful in the present inventive subject matter, and which are the preferred therapeutic active ingredients, include, without limitation, tryptans, oxycodone, morphines, hydrocodone, aspirin, acetaminophen, ibuprophen and mixtures thereof.

Another preferred active material can be selected from the class of prophylactic, abortive or analgesic drugs used to treat migraines. Migraines are defined as headaches that last 4 to 72 hours wherein the patient experiences moderate to severe cranial throbbing. Migraines are also associated with nausea, vomiting, or sensitivity to light, sound or smell.

For prophylactic treatment of migraines, .beta.-blockers, calcium channel blockers, tricyclic antidepressants, or anticonvulsants can be used. Examples of drugs indicated for prophylactic treatment include amitriptyline, methysergide, popranolol, valproate, and verapamil.

For abortive treatment of migraines serotonin receptor activators such as eletriptan, ergotamine, naratriptan, rizatriptan benzoate, sumatriptan succinate, and zolmitriptan can be used. Ergot alkaloid derivatives such as ergoamine tartrate and dihydroergotamine are also effective. Dopamine antagonist anti-emetics such as metoclopramide and prochlorperazine while indicated for the treatment of nausea, can also be used even if nauseau is not prominent.

For analgesic treatment acetaminophen, aspirin, non-asteroidal anti-inflammatory drugs (“NSAID”) and opioids can be used in the present invention.

Yet another preferred active material used in the composition of the present inventive matter is a psychotropic. Psychotropics are used to treat depression, schizophrenia, anxiety disorders, attention deficit order, obsessive compulsive disorder, senile dementia and certain sleep disorders.

The classes of drugs used in treating depression include selective serotonin reuptake inhibitors (“SSRI's”), heterocyclic antidepressants, monoamine oxidase inhibitors (“MAOI's”), serotonergic-noradrenergics, 5-HT.sub.2 antagonists and catecholaminergics. Examples of SSRI'S include fluoxetine HCl, sertraline HCl, paroxetine HCl, and fluvoxamine. Examples of heterocyclic antidepressants include amitriptyline, nortriptyline, imipramine, desipramine, doxepin, trimipramine, clomipramine, protriptyline, amoxapine, and maprotiline. Examples of MAOI's include phenelzine and tranylcypromine. An example of a serotonergic-noradrenergi-cs includes venlafaxine HCl. Examples of 5-HT.sub.2 antagonists include trazadone, nefazodone, and mirtazapine. An example of a catecholaminergics includes bupropion. All examples are non-limiting and it will be understood that psychotropics of the disclosed classes may be used with the present inventive subject matter.

For the treatment of anxiety, benzodiazepines may be used with the present inventive subject matter. Specific examples include alprazolam, chlordiazepoxide, clonazepam, clorazepate, diazepam, lorazepam, and oxazepam. However, any class of psychotropic drug indicated for anxiety treatment may be used in the present invention.

For the treatment of insomnia, drugs belonging to the categories of benzodiazepines, imidazopyridines, antidepressants and non-prescription hypnotics may be used with the present inventive subject matter. Examples of benzodiazepines useful for the treatment of insomnia include midazolam, triazolam, oxazepam, temazepam, lorazepam, estazolam, nitrazepam, diazepam, quazepam, flurazepam, zopiclone and clorazepate. An example of an imidazopyridine includes zolpidem and zolpidem tartarate. Examples of antidepressants include amityiptyline and doxepin.

Still yet another preferred active material used in the composition of the present inventive matter is a gastrointestinal therapeutic. Gastrointestinal therapeutics are used to treat gastritis, nausea and vomiting, gastroesophegal reflux disease, colitis, Crohn's disease and diarrhea. Classes of drugs include proton pump inhibitors, histamine H.sub.2 receptor antagonists, terpene analogs, and NSAID'S.

For the treatment of gastritis, drugs such as omeprazole, lansoprazole, ranitidine HCl, famotidine, nizatidine, teprenone, cimetidine, rabeprazole sodium, and sulpiride can be used in the compositions of the present inventive subject matter.

For the treatment of nausea and vomiting, drugs such as ondansetron HCl, granisetron HCl, dolasetron mesylate, and tropisetron may be used.

Another preferred active material used in the compositions of the present invention include cardiovascular therapeutics. Cardiovascular therapeutics treat hypertension, angina, myocardial infarction, congestive heart failure, acute coronary syndrome, edema, ventricular tachycardia, hyperaldosteronism, ventricular arrhythmia, cardiac insufficiency, atrial fibrillation, arterial occlusion, cardiac decompensation, and microcirculation activation.

A related class of cardiovascular therapeutics are cholesterol reducers such as 3-hydroxy-3-methylglutaryl coenzymeA (“HMG-CoA”) reductase inhibitors. HMG-COA inhibitors work by blocking an enzyme used to make cholesterol. Blocking cholesterol thereby treats hypercholesterolemia which is a significant cause of cardiovascular disease.

For the treatment of hypercholesterolemia, drugs such as simvastin, atorvastatin calcium, pravastatin sodium, pravastatin, lovastatin, fluvastatin sodium, cerivastatin sodium can be used in the compositions of the present inventive subject matter.

For the treatment of hypertension, drugs such as amlodipine besylate, losartan potassium, lisinopril, felodipine, benazepril HCl, ramipril, irbesartan, verapamil HCl, bisoprolol fumarate and hydrochlorothiazide, amlodipine and benazepril HCl, clonidine, candesartan, cilexetil, diltiazem, nicardipine, imidapril, trandolapril, eprosartan mesylate, nilvadipine, verapamil HCl, temocapril, prazosin HCl, isradipine, cilazapril, celiprolol, bisoprolol, betazolol HCl, ramipril, nisoldipine, lisinopril, trandolapril, and nisoldipine can be used in the compositions of the present inventive subject matter.

For the treatment of congestive heart failure, drugs such as dioxin, carvedilol, spironolactone, trandolapril, and bisoprolol can be used in the compositions of the present inventive subject matter.

Still another preferred active material used in the composition of the present invention is a therapeutic useful for treating allergic rhinitis. The classes of compounds useful for treating allergic rhinitis include alkylamines, ethanolamines, ethylenediamines, piperazines, phenothiazine, piperidines, and nonsedating compounds.

Among the non-sedating compounds that can be used in the present invention are loratadine, fexofenadine HCl, certirizine HCl, and astemizole. Other drugs which can also be used are fluticasone propionate, mometasone furoate, epinastine, beclomethasone dipropionate, triamcinolone acetonide, budesonide, and azelastine.

Still yet another preferred active material used in the composition of the present invention is a therapeutic useful for treating osteoarthritis or rheumatoid arthritis. Rheumatoid arthritis is defined as non-specific, symmetrical inflammation of the peripheral joints, potentially resulting in progressive destruction of articular and particular structures. Osteoarthritis is characterized by loss of articular cartilage and hypertrophy of bone. Although osteoarthritis is a degenerative bone disease, symptoms associated with rheumatoid arthritis such as inflammation of the joints occur in a patient diagnosed with osteoarthritis. Accordingly, therapeutics treating rheumatoid arthritis can also be administered to an osteoarthritic patient.

Classes of drugs indicated for osteoarthritis and rheumatoid arthritis include cycloxygenase-2 inhibitors, NSAID'S, biologic response modifiers, pyrimidine synthesis inhibitors and hyaluronic acid. Specific examples of osteoarthritis and rheumatoid arthritis therapeutics include celecoxib, diclofenac sodium, rofecoxib, nabumetone, diclofenac sodium and misoprostol, oxaprozin, meloxicam, piroxicam, etodolac, naproxen, hylan G-F 20, leflunomide, tenoxicam, and naproxen sodium.

Another preferred active material used in the composition of the present invention is a therapeutic useful for treating benign prostatic hypertrophy. Benign prostatic hypertrophy is defined as an adenomatous hyperplasia of the periurethral part of the prostrate gland.

Classes of drug useful for the treatment of benign prostatic hypertrophy include alpha blockers, alpha-1 selective adrenoceptor blocking agents and 5-reductase inhibitors. Specific examples of benign prostatic hypertrophy therapeutics include doxazosin mesylate, terazosin HCl, tamsulosin, finasteride, tamsulosin HCl, ethinyl estradiol and levonorgestrel.

Yet another preferred active material used in the composition of the present invention is a drug indicated for the treatment of fungal infections. Classes of drugs indicated for the treatment of fungal infections include synthetic triazole, ergosterol inhibitor, and polyene antifungal. Specific examples of drugs indicated for the treatment of fungal infections are itraconazole, ketoconazole, and amphotericin B.

Still yet another preferred active material used in the composition of the present invention is a anti-convulsant. Anti-convulsants are drugs that prevent or relieve convulsions wherein the convulsions are due to epilepsy, seizure disorders, partial seizure disorders or Huntington's disease. Classes of drugs useful for treating these conditions include gamma-aminobutyric analogs, phenyltriazine, antiepileptic agents, benzodiazepines, polysynaptic response inhibitors, sulfamate-substituted monosaccharides, gamma-amino butyric acid uptake inhibitors and benzamides. Specific examples include carbamazepine, topiramate, and tigabine HCl mixtures thereof combination drugs.

Another preferred active material used in the composition of the present invention is an anti-herpetic. Anti-herpetics are used to treat infections from the varicella-zoster virus. Classes of drugs useful for treating herpes include synthetic purine nucleoside analogs, nucleoside analogs, and antiviral agents. Specific examples include acyclovir, valacyclovir HCL and famcyclovir mixtures thereof combination drugs.

Yet another active material used in the compositions of the present invention are anti-diarrheal therapeutics. Anti-diarrheal therapeutics treat the condition of diarrhea whether it is symptomatic of the disorder itself wherein diarrhea is a condition that occurs when a mammal has a low amount of stool in a bowel movement. Diarrhea results mainly from excess fecal water in the bowel of the mammal. Specific examples of anti-diarrheal therapeutics include loperamide HCl, diphenoxylate, codeine phosphate, camphorated opium tincture.

Further preferred nutritional active materials useful in the present inventive subject matter include, without limitation, calcium-containing materials such as calcium carbonate, vitamins, minerals, herbals, spices and mixtures thereof.

Examples of vitamins that are available as active ingredients include, without limitation, vitamin A (retinol), vitamin D (cholecalciferol), vitamin E group (a-tocopherol and other tocopherols), vitamin K group (phylloquinones and menaquinones), thiamine (vitamin B.sub.1), riboflavin (vitamin B2), niacin, vitamin B.sub.6 group, folic acid, vitamin B.sub.12 (cobalamins), biotin, vitamin C (ascorbic acid), and mixtures thereof. The amount of vitamin or vitamins present in the final encapsulated product of the present inventive subject matter is dependent on the particular vitamin and is generally the United States' Department of Agriculture Recommended Daily Allowances (USRDA) for that vitamin. For example, if vitamin C is the active ingredient and the encapsulated product is being used in a confectionery or chewing gum targeting adults, the amount of vitamin C in the encapsulated product would be 60 milligrams, which is the USRDA of vitamin C for adults.

Examples of minerals that are available as active ingredients include, without limitation, calcium, magnesium, phosphorus, iron, zinc, iodine, selenium, potassium, copper, manganese, molybdenum and mixtures thereof. As is the case with vitamins, the amount of mineral or minerals present in the final encapsulated product of the present inventive subject matter is dependent on the particular mineral and is generally the USRDA for that mineral. For example, if iodine is the active ingredient and the encapsulated product is being used in a confectionery or chewing gum targeting adults, the amount of iodine in the encapsulated product would be 150 micrograms, which is the USRDA of iodine for adults.

Examples of herbals that are available as active ingredients include, without limitation, echinacea, peppermint, licorice, goldenseal, panax pseudoginseng, grapeseed extract, bilberry, kava, ginko biloba, panax quinquefolium, Siberian ginseng, St. John's wort, bromelian, guglupids, hawthorn, garlic, ginger, angelica species, dandelion, goldenseal, and mixtures thereof. Further, examples of spices that are available as active ingredients include, without limitation, mustard, dillweed, cinnamon, garlic, black pepper, onion, sage, oregano, basil, cream of tartar, targon, cayenne pepper, red pepper, and mixtures thereof. This list of herbals and spices is for exemplary purposes and is not meant to be construed as limiting the inventive subject matter thereto.

In some aspects, the active material may be present in a high dose. As described herein, “high dose” represents a dosage from about 450 mg to about 2000 mg per unit dosage to be administered to a patient. In some aspects, the high dose may represent from about 450 mg to about 2000 mg; or from about 450 mg to about 1200 mg; or from about 500 mg to about 1500 mg; or from about 500 mg to about 1200 mg; or from about 600 mg to about 1500 mg; or from about 600 mg to about 1200 mg. Such high dose actives may be selected from a variety of therapeutic categories, and include, but not limited to the following: an analgesic; an anti-inflammatory; an antibiotic; an antiviral; an anti-irritability; a mineral, or a nutritional supplement, etc. Examples of specific drugs include: aspirin, acetaminophen, naproxen, balsalazide; mesalamine; ampicillin, amoxicillin; clavulanate; azithromycin, clarithromyin, abacavir, lamivudine, acyclovir, atazanavir, efavirenz, fosamprenavir, nelfinavir, ribavirin, saquinavir, and valacyclovir, or a combination thereof. Other examples include: calcium carbonate, glucosamine, chondroitin, Vitamin C, guaifenesin, magnesium hydroxide, caffeine, loratadine, ibuprofeb, pseudoephedrine, diphenhydramine, chlorpheniramine maleate, cimetidine, ranitidine, famotidine, benzocaine, hexylresorcinol, zinc acetate, zinc gluconate, naproxen, naproxen sodium, codeine phosphate, hydrocodone, brompheniramine maleate, docusate sodium, bisacodyl, sennoside, multivitamins (vitamin A, B1, B2, B6, B12), Vitamin E, alone or in combination with another active agent. In some aspects, these active ingredients are encapsulated or coated with a functional or nonfunctional coating, which coated and/or encapsulated ingredients are then used in making the rapid melt compositions. Some nonlimiting examples include: encapsulated glucosamine, chondroitin, encapsulated phenylephrine, encapsulated acetaminophen, encapsulated vitamin C, encapsulated guaifenesin, encapsulated aspirin, calcium carbonate, magnesium hydroxide, or a mixture thereof. Examples of functional coating include enteric coating, pH-dependent coating, sustained release coating. Examples of nonfunctional coating include film coating and sugar coating.

Many of the active material listed above have unpalatable tastes. Taste-masking of compositions with those unpalatable active materials is well-known in the art. The use of flavors and sweeteners to mask the unpalatability of the active materials is also well-known. Thus, other materials which can be incorporated into the rapid-melt composition of the present inventive subject matter include flavors, colors and sweeteners. A distinct feature of the inventive rapid-melt, compositions is that they exhibit excellent taste characteristics. Importantly, it is possible to incorporate high levels of flavors, sweeteners and other taste-masking agents, making the compositions more palatable when undesirable tastes accompany the active materials.

Flavors maybe chosen from natural and synthetic flavor liquids. Flavors useful in the present inventive compositions include, without limitation, volatile oils, synthetic flavor oils, flavoring aromatics, oils, liquids, oleoresins or extracts derived from plants, leaves, flowers, fruits, stems and combinations thereof A non-limiting list of examples include citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral, i.e., alphacitral (lemon, lime), neral, i.e., betal-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), and mixtures thereof.

Further examples of flavors useful in the inventive compositions include, without limitation, beef flavorings, chicken flavorings, rice flavorings, lamb flavorings, pork flavorings, seafood flavorings, and mixtures thereof.

The sweeteners may be chosen from the following non-limiting list: flucose (corn syrup), dextrose, invert sugar, fructose, and mixtures thereof; saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol, and the like. Also contemplated are hydrogenated starch hydrolysates and synthetic sweetener 3,6-dihydro-6-methyl-1-1-1,2,3-oxath-iazin-4-one-2,2-dioxide, particularly the potassium salt (acesulfame-K) and sodium and calcium salts thereof. Other sweeteners may also be used.

The rapid-melt compositions of the present inventive subject matter may also be coated in order to facilitate handling of the compositions. Coatings well-known in the art are useful for keeping the compositions from melting prior to being administered to a patient in need of an active material. By coating the compositions, the composition will maintain its state while being handled and will melt when inserted into a patient's mouth.

The present inventive subject matter also contemplates a method of preparing a rapid-melt composition. A preferred method involves the steps of: melting at least one binder having a melting point about 25 to 90° C. with a salivating agent to form a mixture; mixing an active material with the lipid material to form an active mixture; mixing a diluent/bulking material with said active material to form a final mixture; and molding the final mixture into the composition. The method of the present inventive subject matter also contemplates adding other materials to the final mixture prior to molding into the rapid-melt composition. Other materials which may be added to the final mixture prior to molding include, without limitation, flavors, colors, sweeteners, and mixtures thereof.

The amount of binder melted with the salivating agent is from about 10% to about 70% by weight of the final composition. Preferably, the amount of binder is from about 10% to about 50% by weight. More preferably the binder is present from about 15% to about 30% by weight. Likewise, the amount of salivating agent melted in the first step of the method is from about 0.2% to about 0.5% by weight of the final composition. Preferably, the amount of salivating agent is from about 0.3% to 0.4% by weight of the composition.

However, it should be recognized that the composition may be prepared by a variety of methods well-known by those of ordinary skill in the art. Such processes may be used on a batch or continuous process format and would involve melting the binders and uniformly blending them for suitable periods of time prior to adding the salivating agent. Once these two components have been blended together, the further components may be added either together or sequentially until a uniform mixture is obtained. The Uniform mixture may be poured into a mold, cast into preformed shapes, or stamped into the final products. Clearly, other tableting techniques are contemplated to be used herein.

In a preferred embodiment, the rapid-melt products of the present inventive subject matter are formed via compression of the ingredients. The compression of the ingredients into rapid-melt products may take place in a conventional compression or tableting machine such as a punch and die machine. In addition, the punches used in the punch and die machine may be modified with various materials to limit the formation of a film on the product when the same is punched into shape. One such modification would be to make the punch tips from a copper-beryllium alloy. The use of the copper-beryllium alloy on the tips of the punch, as well as blowing cold low-humidity air on the punch and dies before filling will aid in the reduction of film formation on the products.

Further, additional external lubrication could be added to the punch and die machine while forming the products. The external lubrication may be in the form of a powder lubricant applied via electrostatic method, or the external lubricant may be a liquid lubricant which is applied via conventional jet spraying techniques. In any of the above situations, the film formation during compression will be largely negated.

The binders present in the inventive rapid-melt formulations provide proper binding for the components of the formulation when formed by compression, thus no additional binders or other ingredients are needed. In other words, the binders already present in the inventive products provide enough binding characteristics that no additional binders are needed for the compression step. The fats and emulsifiers acting as the binding agents help form granules that impart flow and compression characteristics in the products.

In a particularly preferred embodiment, after the inventive rapid-melt product has been compressed, the compressed product is exposed to an elevated temperature. The conventional way to expose the compressed rapid-melt product is to employ a conveyor belt on which the compressed rapid-melt product is placed. The conveyor belt then passes through a heating zone, in which heat or hot air is applied to the compressed rapid-melt product. The interior of the compressed product is preferably not heated as much as the exterior of the compressed product. The heat or hot air heats the product or the surface of the product to a temperature of 40 to 60° C. for a period of 1 to 10 minutes. Preferably, the compressed rapid-melt product is heated to a temperature of 45 to 55° C. for a period of 2 to 5 minutes.

Conventional processes may be employed in order to heat the compressed rapid-melt products, with such conventional processes including, but not limited to, a conventional oven, a high voltage heat lamp, a microwave heating element, or the like. If a conventional conveyor belt is used in the heating step, preferably the conveyor will be a stainless steel screened type of conveyor. This will allow the heat to be applied to the product from both the top and the bottom.

In this preferred heating step, the compressed product is slightly heated, causing the emulsifier/fat system to soften or melt within the product. This melting results in the semi-liquid binding system changing its configuration in which the void spaces are filled by the softened or melted emulsifier/fat system present in the product. After the compressed product has been sufficiently heated, the product is cooled to room temperature. Even though the compressed product reaches room temperature relatively quickly, it takes the binding system several hours to return to its original form. This is due to the polymorphism of the emulsifier/fat system. During this time, the weak binding system (due to the relatively poor binding characteristics of the components) is converted to a bonding system between the particles in the compressed product. In this way, the fats and emulsifiers which may be considered weak binders when the compressed rapid-melt product is first granulated and compressed, the fats and emulsifiers now become a much stronger bonding system.

Optionally, the heating step of the inventive process may be done under vacuum, thus enhancing the bonding of the particles by the fat/emulsifier system.

One physical characteristic of the compressed rapid-melt product that is changed due to the bonding of the particles by the melted fat/emulsifier system is the friability of the compressed product. Due to the relatively weak binding characteristics of the fats and emulsifiers, the compressed rapid-melt product may be friable when first compressed. By surface heating the product and converting the binding system to a bonding system, the compressed product has a much higher integrity which allows it to be easily packaged. In other words, the tablet's friability has decreased significantly from very high to almost nothing. The tablet has a high integrity that is suitable for packaging in any form, including large bottles, and the stability of the compressed product is very good.

In a further preferred embodiment of the present inventive subject matter, the active ingredient is added to the compressed rapid-melt composition during the lubrication step of the process. That is, the active ingredient is added to the mixture at the same time that the lubricants are added to the mixture. By adding the active ingredient with the lubricants, the active ingredient is not exposed to the elevated temperatures used to melt the fats and emulsifiers. The lack of exposure to the higher temperature required to melt the fats and emulsifiers helps keep the integrity of the active ingredients intact, meaning that it is less likely for the active ingredients to decompose due to the elevated temperatures.

In addition to the fats and emulsifiers in the composition acting as lubricants (as well as binders), other lubricants may be added in order to enhance lubrication. Such lubricants may be water-soluble or non-water-soluble. Non-limiting examples of such lubricants include magnesium stearate, calcium stearate, talc, starches, silicon dioxide, water soluble lubricants and mixtures thereof. The lubricant may be present in an amount from about 0.1% to about 5%. In some aspects, the lubricant may be present in an amount from about 0.2% to about 3%. In another aspect, the lubricant may be present in an amount from about 0.2% to about 2%.

As stated previously, it is an important aspect of the present inventive subject matter that the compressed rapid-melt product disintegrates quickly in the mouth of the mammal. Preferably, the compressed rapid-melt product disintegrates in less than 20 seconds of being placed in the mammal's mouth, preferably within 10 seconds, and more preferably within 7 seconds. In order to maintain this desired property, it is necessary to compress the components using a low compression force.

It is well-known in the art of compression that tablets are formed by using hard granules prepared by conventional processes, i.e., wet or dry granulation. In all of the conventional processes, strong binders are used to bind the granules and provide good compression and hard tablets when high compression forces are used. Thus, Applicant has found that using the low compressive forces to traditionally-prepared granules results in a compressed product that tend to be friable and fragile.

In a preferred embodiment of the present inventive subject matter, the granules may be prepared with less binding agent than is normally required. In general, the binding agent may be present only in enough amounts to convert the granular powders into the proper form for flowing within the compression machine. Applicant has determined that if granules prepared with less than the required amount of binding agent are then mixed with a bonding agent prior to compression with the low compressive pressures, the resultant product has much improved friability and is able to be handled and packaged more easily than those products prepared by the conventional method of tableting, while still maintaining the requisite disintegration time in the mouth of the user.

The bonding agent promotes good bonding between the particles of the compressed product, thus enhancing the integrity of the compressed product. The bonding agent does so by helping reduce the porosity, i.e. increase the density, in the compressed rapid-melt product and creating close bonds between the particles in the compressed rapid-melt products.

Typical bonding agents include, without limitation, polyethylene glycols in solid form (1450-3000 or more), monoglycerides (40-90% glycerides of vegetable or animal fats), acetylated monoglycerides, hydrocolloidal gums, other emulsifiers or fats and mixtures thereof. The amount of bonding agent present in the inventive subject matter is from 0.1 to (5) to 30% by weight. Preferably, the amount of bonding agent is 0.25 (10) to 15% by weight.

Optionally, the compressed rapid-melt products prepared by this embodiment may be subjected to a heat treatment to further enhance the bonding as is discussed above. In particular, the compressed product is exposed to an elevated temperature. The conventional way to expose the compressed rapid-melt product is to employ a conveyor belt on which the compressed rapid-melt product is placed. The conveyor belt then passes through a beating zone, in which heat or hot air is applied to the compressed rapid-melt product. The heat or hot air heats the product to a temperature of 40 to 60° C. for a period of 1 to 10 minutes. Preferably, the compressed rapid-melt product is heated to a temperature of 45 to 55° C. for a period of 2 to 5 minutes.

Conventional processes may be employed in order to heat the compressed rapid-melt products, with such conventional processes including, but not limited to, a conventional oven, a high voltage heat lamp, a microwave heating element, or the like. If a conventional conveyor belt is used in the heating step, preferably the conveyor will be a stainless steel screened type of conveyor. This will allow the heat to be applied to the product from both the top and the bottom.

In this preferred heating step, the compressed product is slightly heated, causing the emulsifier/fat system to soften or melt within the product. This melting results in the semi-liquid binding system changing its configuration in which the void spaces are filled by the granules present in the product. The interior of the compressed product is preferably not heated as much as the exterior of the compressed product.

After the compressed product has been sufficiently heated, the product is cooled to room temperature. Even though the compressed product reaches room temperature relatively quickly, it takes the binding system several hours to return to its original form. This is due to the polymorphism of the emulsifier/fat system. During this time, the weak binding system (due to the relatively poor binding characteristics of the components) is converted to a bonding system between the particles in the compressed product. Whereas the fats and emulsifiers are weak binders when the compressed rapid-melt product is first granulated and compressed, the fats and emulsifiers now become a much stronger bonding system.

Optionally, the heating step of the inventive process may be done under vacuum, thus enhancing the bonding of the particles by the fat/emulsifier system.

The rapid-melt compositions of the present inventive subject matter produced by the above methods have increased product integrity and stability. The compositions are “storage stable”, meaning that the compositions are stable in the absence of special handling procedures. The inventive compositions are stable both prior to packaging and after packaging. Importantly, the inventive compositions maintain their stability and integrity without refrigeration and without humidity controls being implemented during handling, packaging and storing of the products. Additionally, since the compositions exhibit increased integrity and stability, the compositions can be used in most of the current economical packages suitable for a global environment. Further, high temperatures are not needed when processing the inventive compositions. The only heat that needs to be used during processing is to melt the binder prior to mixing with the other elements.

The compositions of the present invention can be appropriately used to make rapid melt tablets, chew tablets, or flashbeads. As used herein, “rapid melt tablet” is a compressed tablet that melts or disintegrates in the mouth in the presence of saliva without having to be chewed. Preferably, a rapid melt tablet when placed in the mouth melts within about 30 seconds, preferably about 20 seconds, more preferably about 10 seconds, and most preferably about 5 seconds. The rapid melt tablets preferably contains super emulsifiers to effect the rapid melt property.

A typical process for making rapid melt tablets includes forming a first premix (premix 1) of diluent/bulking materials, flavor (if any), and sweetener (if any), forming a second premix (premix 2) of the binder(s) and emulsifier(s), and forming a third premix (premix 3) of the active ingredient and diluent/bulking materials. Premix 3 and premix 2 are the blended together; then premix 1 is then added and blended. Lubricants, if any, are then added to the blend (of premix 1, 2, 3) and further blended. The final blend is then compressed into tablets using, e.g., a tablet press.

As used herein, “chew tablet” refers to compressed tablet that, when placed in the mouth, must be chewed to disintegrate. Generally, the chew tablet do not melt in the mouth without being chewed. The chew tablet preferably contains emulsifier(s) having a high melting point of greater than body temperature, preferably greater than about 40° C., more preferably greater than 45° C., and most preferably about 40-60° C. Also, in this case, the emulsifier are also preferably used as binders. During processing of chew tablets, powder lubricants, such as magnesium stearate, silicon dioxide, and glidant talc, and diluent/bulking material(s) are preferably added to the mixture last, just before tablet compression. The preferred emulsifier for chew tablets include glycerides, lecithin, or combinations thereof. The most preferred emulsifers are diglycerides, monoglycerides, acetylated monoglycerides, lecithin, or combinations thereof.

A typical process for making rapid melt tablets includes first mixing and heating the emulsifiers to a molten mixture, which is then added to the active ingredient under continuous stirring. The mixture is then cooled and, preferably, milled to produce granules having good granular flow. Powder lubricants, diluent/bulking material(s), and sweetener (if any) are then added to the granules and blended. The final blend is compressed into tablets using, e.g., a tablet press.

As used herein, “flashbead” refers to a granule or bead that melts or disintegrates in the mouth in the presence of saliva without having to be chewed. Preferably, a rapid melt tablet when placed in the mouth melts within about 30 seconds, preferably about 20 seconds, more preferably about 10 seconds, and most preferably about 5 seconds. Generally, the flashbead formulation cannot be compressed into tablet form without defects, such as pitting, chipping, or broken tablets. The flashbead preferably contains binder(s) having a low melting point of about 20-90° C., preferably less than body temperature, and more preferably less than about 30-40° C. The preferred binder for flashbeads is cocoa butter. The preferred emulsifier for flashbeads are polysorbate 80, sodium lauryl sulfate, or combinations thereof.

A typical process for making flashbeads includes first mixing the binder(s) and emulsifier(s) in solution or liquid phase. Active ingredients and diluent/bulking material(s) are then added to the binder/emulsifier and blended and granulated. This final mixture is then extruded and spheronized to produce the desired granules or beads.

In an embodiment, instead of including an active ingredient in the flashbeads, the flashbeads can contain a placebo. In this case, the active ingredient can be replaced by a diluent/bulking material. In use, the flashbeads are co-administered with granules or beads containing an active ingredient, where the flashbeads serve a carrier that allows the active ingredient to dissolve in the mouth and be swallowed without the administration of water. In this case, the flashbeads itself do not contain the active ingredient. Rather, the active ingredient is contained in the granules or beads that are co-administered with the flashbeads.

In another embodiment, flashbeads can be made using super emulsifiers. This embodiment results in a composition similar for that of the rapid melt tablet above. However, instead of pressing the composition into a tablet, the final mix is extruded and spheronized to form flashbeads.

The compressed form of the present invention compositions can be manufactured using conventional press or punch. The compression force used in the tabeleting process is generally between about 15 KN to about 40 KN. The compression force can be adjusted based on the size, shape of the tablet, and the desired hardness of the tablet. For “rapid melt tablet,” the compression force is typically at about 18 to about 32 KN. For “chew tablet,” the compression force is typically at about 20 to about 25 KN.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in those examples. The amount of active ingredients per unit dosage form can be changed to accomodate multiple strengths of dosage form. The amount of excipients may remain the same or adjusted slightly to accommodate formulation-related parameters such as flowability, compressibility, etc. All percentages are given in weight percent, unless otherwise noted and are based on 100% by weight of the final compositions.

### EXAMPLE 1

### Preparation of Compressed Rapid-Melt Product Containing Chondroitin and Glucosamine

4.51% cocoa butter, 9.01% sorbitan monostearate, 0.45% lecithin, 0.36% polysorbate 20, 0.45% sodium lauryl sulfate, 0.02% color agent, 0.02% sucralose, 1.62% citric acid and 7.03% chondroitin sulfate were mixed in a heating vessel. The mixture was stirred and heated to a temperature of 130° F. The mixture was maintained at the 130° F. temperature while 34.05% of xylitol powder was added under continual stirring, along with 2.53% powdered flavors pre-blended with 9.01% xylitol powder.

Upon complete blending of the above components, the mixture was transferred to wax paper and cooled to 41° F. for 30 minutes. Once completely cooled, the mixture was milled using a colloidal mill with a #16 screen.

In the meantime, 24.61% encapsulated glucosamine, 4.51% maltodextrin, 0.45% silicon dioxide, 0.90% magnesium stearate, 0.45% additional powdered flavors, and 0.02% color agent were mixed, then passed through a #30 mesh.

After sieving the above mixtures, the two mixtures were blended together and compressed in a conventional compression tableting machine.

In this example, one active ingredient (chondroitin) was added in the emulsifier melting step, while another active (glucosamine) was added during the lubrication step.

### EXAMPLE 2

### Preparation of Compressed Rapid-Melt Product Containing Glucosamine

9.5% acetylated monoglycerides, 17.7% hydrogenated vegetable oil and 3.0% monoglycerides were mixed in a suitable vessel and heated to 150° F. to melt the fats. Meanwhile, 68.7% glucosamine hydrochloride powder was pre-blended with 0.8% aspartame and 0.3% sodium laurel sulfate. Once the fats had completely melted, the pre-blended glucosamine hydrochloride mixture was added to the vessel. The fats/glucosamine mixture was them mixed well at 150° F.

Upon complete blending of the above components, the mixture was transferred to wax paper and cooled to 41° F. for 30 minutes. Once completely cooled, the mixture was milled using a colloidal mill with a #16 screen. The resultant product was then compressed in a conventional compression tableting machine.

### EXAMPLE 3

### Preparation of Compressed Rapid-Melt Product Containing Calcium

18.80% hydrogenated vegetable oil, 9.68% monoglycerides, 0.48% polysorbate 80, 0.06% sodium lauryl sulfate and 0.02% color agent were mixed and heated in a suitable vessel. The mixture was heated to 130° F. for 10 minutes until the components melted into a solution. 48.76% dextrose powder was added to the mixture under constant stirring along with a pre-blended mixture of 0.04% cooling agent and 1.55% flavors in 12.1% dextrose powder.

Upon complete blending of the above components, the mixture was transferred to wax paper and cooled to 41° F. for 30 minutes. Once completely cooled, the mixture was milled using a colloidal mill with a #16 screen.

In the meantime, 0.28% aspertame, 1.22% powdered flavors, 1.47% silicon dioxide, 1.22% magnesium stearate, 0.6% polyethylene glycol, 3.7% maltodextrin and 0.02% color agents were mixed and passed through a #30 mesh.

After sieving the above mixtures, the two mixtures were blended together and compressed in a conventional compression tableting machine

### EXAMPLE 4

### Preparation of Cellulose-Containing Compressed Rapid-Melt Product with a Bonding Agent

A 5.0% hydrocolloidal gum solution in water was prepared. The solution was mixed well and set aside until free from lumps. In the meantime, 73.40% mannitol powder was blended with 24.6% microcrystalline cellulose and 0.21% color agents. After mixing an appropriate time, the gum solution was added to the mixture in small amounts. Just enough gum solution was added to form small lumps or aggregates. The wet aggregates were passed through a #8 screen.

After sieving, the granules were placed on trays and allowed to dry using air heated to greater than 150° F. Once completely dry, the granules were ground to a #40 mesh size. The granules were then loaded into a conventional tableting machine and tablets were produced.

The resultant tablets were of sufficient hardness and provided proper liquification in the mouth.

### EXAMPLE 5

### Preparation of Bonded Rapid-Melt Product

Mannitol granules were prepared by mixing 89.00% mannitol with 10.00% microcrystalline cellulose. The mannitol and microcrystalline cellulose were granulated with 1.00% polyvinyl pyrrolidone.

Following granulation of the mannitol, 77.98% of the above mannitol granules were mixed with 0.20% sucrose, 0.05% sodium lauryl sulphate and 0.07% carboxamide. 1.85% suitable flavors and 6.98% encapsulated active ingredients were added to the mixture. Following proper mixing of the mannitol granules with the remaining above ingredients, 10.20% bonding agent, in this case 10.00% sorbitan monostearate and 0.20% Tween 80, along with 1.00% crosspovidone, 0.50% tale, and 0.75% magnesium stearate were added to the mixture. The final mixture was then tableted using 0.75-inch punches.

The resulting product exhibited good granular flow as well as good hardness of the final product. The product was able to be handled and packaged in a conventional manner. The product melted within 25 seconds of being placed in the mouth of a mammal.

### EXAMPLE 6

### Preparation of Bonded Rapid-Melt Product

Mannitol granules were prepared by mixing 89.00% mannitol with 10.00% microcrystalline cellulose. The mannitol and microcrystalline cellulose were granulated with 1.00% polyvinyl pyrrolidone.

Following granulation of the mannitol, 79.85% of the above mannitol granules were mixed with 0.20% sucrose, 0.05% sodium lauryl sulphate and 0.07% carboxamide. 1.85% suitable flavors and 6.98% encapsulated active ingredients were added to the mixture. Following proper mixing of the mannitol granules with the remaining above ingredients, 5.00% bonding agent, in this case sorbitan monostearate, and 5.00% talc as a lubricant, along with 1.00% crosspovidone were added to the mixture. The final mixture was then tableted using 0.75-inch punches.

The resulting product exhibited good granular flow as well as good hardness of the final product. The product was able to be handled and packaged in a conventional manner. The product melted within 10 seconds of being placed in the mouth of a mammal.

### EXAMPLE 7

### Preparation of a Non-Bonded Rapid-Melt Product

Mannitol granules were prepared by mixing 89.00% mannitol with 10.00% microcrystalline cellulose. The mannitol and microcrystalline cellulose were granulated with 1.00% polyvinyl pyrrolidone.

Following granulation of the mannitol, 89.95% of the above mannitol granules were mixed with 0.20% sucrose, 0.05% sodium lauryl sulphate and 0.07% carboxamide. 1.85% suitable flavors and 6.98% encapsulated active ingredients were added to the mixture. Following proper mixing of the mannitol granules with the remaining above ingredients 1.00% crosspovidone was added to the mixture. The final mixture was then tableted using 0.75-inch punches.

The resulting product exhibited good granular flow; however, the product was very brittle and easily crumbled when pressed between one's fingers. The product would not have been easily handled or packaged.

### EXAMPLE 8

### Preparation of Compressed Rapid-Melt Tablets Containing Chondroitin Sulfate 400 mg and Glucosamine HCl 500 mg

Mono and diglycerides (Durem 117) 7.00%, acetylated monoglycerides (Myvacet) 7.00%, (both as salivating agents) 0.05% color agent, 0.2% sucralose, 2.25% citric acid (also as a salivating agent), 51.28% encapsulated glucosamine, and 400 mg chondroitin sulfate were mixed in a heating vessel. The mixture was stirred and heated to a temperature of 130° F. The mixture was maintained at the 130° F. temperature while citric acid 2.25% was added under continual stirring, along with 1.0% powdered flavors. Encapsulated glucosamine comprised of glucosamine HCl, distilled mono and diglycerides as binders and emulsifiers and silicon dioxide as lubricant. Encapsulation was accomplished by heating the distilled mono- and di-glycerides to about 70 C and adding glucosamine with thorough mixing at about 82 C. The mixture was then allowed to cool slowly while adding the lubricant. At about 40 C, taste-masked encapsulated glucosamine was

Upon complete blending of the above components, the mixture was transferred to wax paper and cooled to 41° F. for 30 minutes. Once completely cooled, the mixture was milled using a colloidal mill with a #16 screen.

In the meantime, 3.55% dextrose, 0.5% silicon dioxide, 0.50% magnesium stearate, 0.45% additional powdered flavors, and 0.02% color agent were mixed, then passed through a #30 mesh.

After sieving the above mixtures, the two mixtures were blended together and compressed in a conventional compression tableting machine.

### EXAMPLE 9

### Preparation of Compressed Rapid-Melt Tablets Containing Glucosamine HCl 500 mg

The process of Example 8 was followed with the following changes: encapsulated glucosamine HCl comprising 500 mg active and representing 90.5% of the composition. Dextrose monohydrate is at 2.45% by weight of the composition. The preparation was made into bead forms which were then compressed into tablets.

### EXAMPLE 10

### Preparation of Compressed Rapid-Melt Tablets Containing Calcium Carbonate 625 mg-1250 mg

Mono and diglycerides (Durem 117) 12.00%, acetylated monoglycerides (Myvacet) 7.00%, 0.03% color agent, 0.25% sucralose, distilled monoglycerides, 5.0%, polyethyleneglycol 3350, 4.0%, and 62.5% calcium carbonate were mixed in a heating vessel. The mixture was stirred and heated to a temperature of 130° F. The mixture was maintained at the 130° F. temperature while 1.0% powdered flavors were added under continual stirring.

Upon complete blending of the above components, the mixture was transferred to wax paper and cooled to 41° F. for 30 minutes. Once completely cooled, the mixture was milled using a colloidal mill with a #16 screen.

In the meantime, 1.0% maltodextrin, 4.2% microcrystalline cellulose, 0.5% silicon dioxide, 0.50% magnesium stearate, 1.5% additional powdered flavors, and 0.02% color agent were mixed, then passed through a #30 mesh.

After sieving the above mixtures, the two mixtures were blended together and compressed in a conventional compression tableting machine.

The above formulation is dose-proportional. Thus, 1250 mg calcium carbonate high-dose product can be made by using the above formulation with ingredients adjusted accordingly.

### EXAMPLE 11

### Preparation of Compressed Rapid-Melt Tablets Containing Acetaminophen 650 mg and Phenylephrine 10 mg

7.0% mono- and di-glycerides, 7.0% acetylated monoglycerides, 0.1% polysorbate 80, 0.1% sodium lauryl sulfate, 0.02% color agent, and 59.1% encapsulated acetaminophen, 1.82% encapsulated phenylephrine, and 0.02% sucralose were mixed in a heating vessel. The mixture was stirred and heated to a temperature of 130° F. The mixture was maintained at the 130° F. temperature while 0.20% powdered flavors were added under continuous stirring.

Upon complete blending ofthe above components, the mixture was transferred to wax paper and cooled to 41° F. for 30 minutes. Once completely cooled, the mixture was milled using a colloidal mill with a #16 screen.

In the meantime, 6.98% maltodextrin, 7.0% polyvinylpyrrolidone, 1.0% magnesium stearate, 0.45% additional powdered flavors, and 0.01% color agent were mixed, then passed through a #30 mesh.

After sieving the above mixtures, the two mixtures were blended together and compressed in a conventional compression tableting machine.

Alternatively, the sieved mixture is made into granules or beads (without compression) for administration or for further processing.

### EXAMPLE 12

### Preparation of Compressed Rapid-Melt Tablets Containing Acetaminophen 160 mg

Polysorbate-80 (0.1%), sodium lauryl sulphate (0.05%) and PEG 8000 (1.5%) 0.02% color agent, and 25% encapsulated acetaminophen, 0.75% sucralose were mixed in a ( ) vessel. (The mixture was stirred and heated to a temperature of 130° F. The mixture was maintained at the 130.degree.) F. temperature while 0.20% powdered flavors were added under continuous stirring.

Upon complete blending of the above components, the mixture was transferred to wax paper and cooled to 41° F. for 30 minutes. Once completely cooled, the mixture was milled using a colloidal mill with a #16 screen.

In the meantime, 37.7% dextrose monohydrate, 7.5% polyvinylpyrrolidone, 0.6% magnesium stearate, 0.45% additional powdered flavors, and 0.01% color agent were mixed, then passed through a #30 mesh.

After sieving the above mixtures, the two mixtures were blended together and compressed in a conventional compression tableting machine.

Alternatively, the sieved mixture is made into granules or beads (without compression) for administration or for further processing.

### EXAMPLE 13

### Preparation of Compressed Rapid-Melt Tablets Containing Acetaminophen 500 mg

The process of Example 12 was used with the following changes: encapsulated acetaminophen (39.7%); dextrose monohydrate 48%; PEG 8000 (2.0%); polyvinylpyrrolidone (Polyplasdone XL-10) (7.5%).

### EXAMPLE 14

### Preparation of Compressed Rapid-Melt Tablets Containing Acetaminophen 81 mg

The process of Example 12 was used with the following changes: encapsulated acetaminophen (23.62%); dextrose monohydrate (53%); PEG 8000 (1.5%); polyvinylpyrrolidone (Polyplasdone XL-10) (7.5%); citric acid (0.65%).

### EXAMPLE 15

### Preparation of Compressed Rapid-Melt Tablets Containing Guaifenesin 100 mg Beads

The process of Example 12 was used with the following changes: encapsulated guaifenesin (31%), wherein the encapsulation was 54%; dextrose monohydrate (42%); PEG 3350 (2.1%); polysorbate 80 (0.2%) sodium lauryl sulfate (0.0035%); polyvinylpyrrolidone (Polyplasdone XL-10) (1.4

### EXAMPLE 16

### Preparation of Compressed Rapid-Melt Tablets Containing Phenylephrine HCl 10 mg

The process of Example 12 was used with the following changes: encapsulated phenylephrine HCl (10.5%); dextrose monohydrate (47%); PEG 8000 (1.0%); polysorbate 80 (0.26%); sodium lauryl sulfate (0.05%); polyvinylpyrrolidone (Polyplasdone XL-10) (4.0%); sodium starch glycolate (3.44%).

### EXAMPLE 17

### Preparation of Compressed Rapid-Melt Tablets Containing Acetaminophen 325 mg and Phenylephrine 5 mg

The process of Example 11 was used with the following changes: encapsulated phenylephrine HCl (1.65%); encapsulated acetaminophen (35.75%); dextrose monohydrate (35%); PEG 8000 (2.0%); polysorbate 80 (0.1%); sodium lauryl sulfate (0.05%); polyvinylpyrrolidone (Polyplasdone XL-10) (7.0%).

### EXAMPLE 18

### Preparation of Compressed Rapid-Melt Tablets Containing Vitamin C 500 mg

The process of Example 12 was used with tbe following changes: encapsulated Vitamin C (91.63%); dextrose monohydrate (3.1%); camuba wax (14%) of the encapsulated Vitamin C composition; mono- and di-glycerides with acetylated monoglycerides (38% of the encapsulated Vitamin C composition); polyvinylpyrrolidone (Polyplasdone XL-10) (4.0%); sodium starch glycolate (3.44%).

### EXAMPLE 19

### Preparation of Compressed Rapid-Melt Tablets Containing Calcium Carbonate 750 mg and Magnesium Hydroxide 135 mg

The process of Example 12 was used with the following changes: calcium carbonate (50%); magnesium hydroxide (9.0%); acetylated monodiglycerides (7.0%); mono- and diglycerides (7.0%); PEG 3350 (3.0%).

### EXAMPLE 20

### Preparation of Acetaminophen 160 mg Rapidmelt

Mannitol granules (152 mg) was sifted through # 24 mesh. Grape flavor (4.9 mg) and sweetener (2.8 mg) were then added. This mixture was set aside as premix 1.

Crospovidone (49 mg), sodium lauryl sulphate (0.7 mg) and sodium starch glycolate (14 mg) were mixed together. Polysorbate 80 (1.05 mg) was slowly added to this mixture which acts as emulsifier. After completely adding the polysorbate 80, the mixture was mixed for 10 minutes, and then sieved through # 35 mesh. This mixture was set aside as premix 2.

Lubricants magnesium stearate (4.20 mg) and silicon dioxide (1.4 mg) were sifted throught #24 mesh.

Mannitol granules (152.71 mg), microcrystalline cellulose (140 mg) and color agents (1.4 mg) were sifted through # 24 mesh. This mixture is transferred to an appropriate blender; and encapsulated acetaminophen (175.84 mg) was added to it and blended for 20 minutes.

Premix 2 was then added to the blender and blended for another 10 minutes. Then the premix 1 was added to the blender and blended for another 15 minutes. The lubricants magnesium stearate (4.20 mg) and silicon dioxide were added to the blender and blended for an additional 5 minutes. The blend is compressed in conventional compression machine.

The resulting blend exhibited good granular flow. The blend was able to be compressed using conventional tablet press. Punch Size: 14 mm; Shape: Round, Lozenge;

Tablet weight: 700 mg, Thickness: 0.18″, Hardness: 3.5 kp, Friability: 0.1%, Disintegration time: 22 seconds, Content uniformity Average: 101.8%, % RSD: 3.3%, Assay Average: 102.7%

Tablets had a smooth mouth feel, tasted good, and dissolved in the mouth in less than about 45 seconds.

Dissolution test was performed for the tablets using a medium of pH 5.8 phosphate buffers, volume: 900 ml, dissolution apparatus II paddle, at speed of 75 rpm. About 100% of the drug was released within 45 minutes.

### EXAMPLE 21

### Preparation of Acetaminophen 80 mg Rapidmelt

The general procedure of Example 20 was followed for the preparation of Acetaminophen 80 mg Rapidmelt. The excipients are adjusted accordingly. The blend was compressed using conventional tablet press. Punch Size: 11 mm; Shape: Round, Lozenge;

Tablet weight: 350 mg, Thickness: 0.13″, Hardness: 4.1 kp, Disintegration time: 48 seconds, Friability: 0.039%.

Tablets had a smooth mouth feel, tasted good, and dissolved in the mouth in less than about 45 seconds.

Dissolution test was performed for the tablets using a medium of pH 5.8 phosphate buffers, volume: 900 ml, dissolution apparatus II paddle, at speed of 75 rpm. 99.2% of the drug released within 45 minutes.

### EXAMPLE 22

### Preparation of Caffeine 40 mg and Taurine 10 mg Rapidmelt

Powder flavor (100.01 mg), sweetner (60 mg), sodium lauryl sulfate (0.65 mg) were added to dextrose monohydrate (210.74 mg). The flavor premix was passed through mesh # 35. The mixture was set aside as premix 1.

Polysorbate 80 (1.30 mg), as an emulsifier, adsorbed on crospovidone (39.00 mg) and sifted through # 24 mesh. Calcium Silicate (6.50 mg) is then added; and the mixture was set aside as premix 2.

Microcrystalline cellulose (32.50 mg) and mannitol granules (210.68 mg) were mixed together and passed through mesh # 35. This mixture was set aside as premix 3.

Sorbitan monostearate (19.50 mg) was passed through # 35 mesh. The premix 3 were added into the appropriate traditional blender together with encapsulated caffeine (100.01 mg) and L-taurine (13.20 mg) and blended for 3 minutes. The premix 2 was then added to the blender and blended for another 3 minutes. Then the premix 1 was added to the blender and blended for an additional 5 minutes. The sifted sorbitan monostearate was then transferred to the blender and blended for another more 4 minutes.

The lubricant Magnesium Stearate (3.90 mg) and glidant Talc (1.30 mg) were sifted one after another through # 35 mesh and added to the blender and blended for an additional 3-5 min.

The resulting blend exhibited good granular flow. The blend was able to be compressed using conventional tablet press. Punch Size: 14 mm; Shape: Round, Lozenge;

Tablet weight: 650 mg, Thickness: 0.14″, Hardness: 4.2 kp

Tablets had a smooth mouth feel, tasted good, and dissolved in the mouth in less than about 45 seconds.

### EXAMPLE 23

### Preparation of Zinc Rapidmelt

Mannitol granules (486.3 mg) was sifted through mesh # 24. Sorbitan monostearate (8.2 mg) was sifted through mesh # 24. The sweetener (2.46 mg), flavor (20.5 mg), sodium lauryl sulfate (0.41 mg) and povidone K-29/32 (5.99 mg) were added to the sorbitan monostearate (the flavor premix).

Zinc gluconate dihydrate (18.34 mg) and milled zinc acetate dihydrate (26.45 mg) were passed through mesh # 24. Polyplasdone premix (polysorbate 80 (3.38%), crospovidone (51.92%), and sodium starch glycolate (44.69%)) (63.17 mg) was passed through #24 mesh.

The lubricants magnesium stearate (4.92 mg), silicon dioxide (0.82 mg) and talc (1.64 mg) were sifted through mesh # 24. Sifted mannitol granules, microcrystalline cellulose (180.8 mg), the flavor premix, sifted zinc gluconate and zinc acetate dehydrate, polyplasdone premix were transferred to an appropriate traditional blender and mixed for 45 minutes. Then the sifted lubricants are transferred to the blender and blended for another 3 minutes.

The resulting blend exhibited good granular flow. The blend was able to be compressed using conventional tablet press. Punch Size: 0.3500″×0.8500″, Shape: Rectangle,

Tablet weight: 820 mg, Thickness: 0.18″, Hardness: 5.0 kp, Friability: 0.02%, Disintegration time: 24 sec, Content uniformity average: 103%, % RSD: 1.1, Assay: 102.9%

Tablets had a smooth mouth feel, tasted good, and dissolved in the mouth in less than about 45 seconds.

### EXAMPLE 24

### Preparation of Zinc Rapidmelt

Zinc acetate dihydrate (36.98 mg), flavor (20.50 mg), povidone (5.99 mg), polyplasdone premix (polysorbate 80 (3.38%), crospovidone (51.92%), and sodium starch glycolate (44.69%)) (63.14 mg), microcrystalline cellulose (180.40 mg), polyethylene glycol 8000 (8.20 mg), sodium lauryl sulfate (0.41 mg), sorbitan monostearate (8.20 mg), sweetener (3.28 mg), and mannitol granules (485.52 mg) were sifted through # 24 screen. These materials were then transferred to the appropriate traditional blender and blended for 45 minutes.

Magnesium stearate (4.92 mg), talc (1.64 mg), and silicon dioxide (0.82 mg) were sifted through #24 Screen; and then added to the blender (along with the ingredients already in the blender) and mixed for 5 minutes. The resulting blend exhibited good granular flow. The blend was able to be compressed using conventional tablet press. Punch Size: 15 mm, Shape: lozenge.

Tablet weight: 820 mg, Thickness: 0.19″, Hardness: 5.7 kp, Friability: 0.1%, Disintegration time: 54 sec, Content uniformity average: 104.6%, % RSD: 2.1, Assay: 103.9%

Tablets had a smooth mouth feel, tasted good, and dissolved in the mouth in less than about 45 seconds.

### EXAMPLE 25

### Preparation of 5-Hydroxytryptamine Receptor Agonist Sumatriptan 25 mg Rapidmelt

Microencapsulated sumatriptan succinate powder (175 mg) was mixed with sweetener (8.2 mg) for 5 minutes. The material was then sifted through # 35 mesh. Microcrystalline cellulose (112.45 mg) and granular Mannitol (50 mg) were added to the sifted material and mixed for 10 minutes. Cross caramellose Sodium (4 mg) were then added to the mixture and mixed well.

Lubricant magnesium stearate (0.35 mg) was sifted through # 60 mesh and added to the above mixture and blended for 10 minutes.

The resulting blend exhibited good granular flow. The blend was able to be compressed using conventional tablet press. Punch Size: 14 mm; Shape: Round, Lozenge; Tablet weight: 350 mg

Tablets had a smooth mouth feel, tasted good tasting, dissolved in the mouth in less than about 45 seconds.

Several batches were prepared. Dissolution test was performed for the tablets using a medium pH 1.2, volume: 900 ml, dissolution apparatus II paddle, at speed of 50 rpm. 97% of the drug released in five minutes.

### EXAMPLE 26

### Preparation of Risperidone 1 mg Rapidmelt

Encapsulated risperidone (210 mg), sodium lauryl sulfate (0.3 mg), sweetening agents (3 mg) and flavors (1.2 mg), mannitol granules (58.2 mg) were transferred to an appropriate blender.

Polysorbate 80 (0.3 mg) was absorbed over crospovidone (24 mg), sieved through mesh #40, and was transferred to the blender and the mixture was blended for 20 minutes.

Lubricant magnesium stearate (2.4 mg), silicon dioxide (0.21 mg) and talc (0.39 mg) were added to the above mixture and blended for additional 5 minutes.

The resulting blend exhibited good granular flow. The blend was able to be compressed using conventional tablet press. Punch Size: 9.0 mm; Shape: Lozenge; Tablet weight: 300 mg

Tablets had a smooth mouth feel, tasted good and dissolved in the mouth in less than about 45 seconds.

### EXAMPLE 27

### Other Preparations of Risperidone Rapidmelt

Risperidone Rapidmelt formulations analogous to that of Example 26 were prepared where the amount of Risperidone per unit dosage form is changed to contain 0.25 mg, 0.5 mg, 2 mg, 3 mg or 4 mg of risperidone. The amount of excipients may remain the same or adjusted slightly to accommodate formulation-related parameters such as flowability, compressibility, etc. The resulting tablets have essentially same characteristic and taste as Risperidone 1 mg.

### EXAMPLE 28

### Preparation of Amlodipine Besylate 10 mg Rapidmelt

Amlodipine besylate (1 mg), sweetening agents (0.4 mg) and flavors (0.4 mg), mannitol granules (50 mg), microcrystalline cellulose (31 mg) were transferred to an appropriate blender.

Polysorbate 80 (0.1 mg) was absorbed over crosspovidone (7 mg), sieved through mesh #40, and then transferred to blender and blended for 20 minutes.

Lubricant magnesium stearate (0.8 mg), silicon dioxide (0.07 mg) and talc (0.13 mg) were added to the above mixture and blended for and additional 5 minutes.

The resulting blend exhibited good granular flow. The blend is able to be compressed using conventional tablet press. Punch Size: 7.0 mm; Shape: Lozenge; Tablet weight: 100 mg

Tablets had a smooth mouth feel, tasted good and dissolved in the mouth in less than about 45 seconds.

### EXAMPLE 29

### Other Preparations of Amlodipine Rapidmelt

Amlodipine Rapidmelt formulations analogous to that of Example 28 were prepared where the amount of amlodipine base per unit dosage form is changed to contain 2.5 mg or 5 mg of amlodipine. The amount of excipients may remain the same or adjusted slightly to accommodate formulation-related parameters such as flowability, compressibility, etc. The resulting tablets have essentially same characteristic and taste as Amlodipine Besylate 10 mg.

### EXAMPLE 30

### Preparation of Olanzapine 5 mg Rapidmelt

Olanzapine (5 mg), sweetening agents (0.5 mg) and flavors (0.4 mg), mannitol granules (44.8 mg), microcrystalline cellulose (39.1 mg) were transferred to an appropriate blender.

Polysorbate 80 (0.01 mg) was absorbed over crospovidone (9 mg), sieved through mesh #40, and transferred to blender and blended for 20 minutes.

Lubricant magnesium stearate (0.8 mg), silicon dioxide (0.07 mg) and talc (0.13 mg) were added to the above mixture and blended for another 5 minutes.

The resulting blend exhibited good granular flow. The blend is able to be compressed using conventional tablet press. Punch Size: 7.0 mm; Shape: Lozenge; Tablet weight: 100 mg

Tablets had a smooth mouth feel, tasted good and dissolved in the mouth in less than about 45 seconds.

Several batches are prepared. Dissolution test was performed for the tablets using a medium pH 6.8 buffers, volume: 900 ml, dissolution apparatus II paddle, at speed of 50 rpm.

90% of the drug was released in 30 minutes.

### EXAMPLE 31

### Other Preparations of Olanzapine

Rapidmelt formulations analogous to that of Example 30 were prepared wherein the amount of olanzapine per unit dosage form is changed to contain 10 mg, 15 mg, or 20 mg of olanzapine. The amount of excipients may remain the same or adjusted slightly to accommodate formulation-related parameters such as flowability, compressibility, etc. The resulting tablets have essentially same characteristic and taste as Olanzapine 5 mg, dissolution data is available only for Olanzapine 5 mg.

### EXAMPLE 32

### Preparation of Loratadine 10 mg Rapidmelt

Loratadine (5 mg), sweetening agents (0.3 mg) and flavors (0.4 mg), sodium lauryl sulfate and mannitol granules (35 mg) were transferred in an appropriate traditional blender.

The lubricants magnesium stearate (0.8 mg), purified talc (0.13 mg) and silicon dioxide (0.07 mg) were passed through # 50 mesh. Polysorbate 80 (0.1 mg) was absorbed on crospovidone (8 mg). This polysorbate 80 on crospovidone was added to polyethylene glycol (1 mg) and transferred to a blender and blended for 20 minutes. Then lubricants then transferred to the blender and blended for another 5 minutes.

The resulting blend exhibited good granular flow. The blend was able to be compressed using conventional tablet press. Punch Size: 7.0 mm; Shape: Lozenge; Tablet weight: 100 mg, Hardness: 2.2 kp,

Tablets had a smooth mouth feel, tasted good and dissolved in the mouth in less than about 45 seconds.

### EXAMPLE 33

### Preparation of Ibuprofen 200 mg Rapidmelt

Sweetner (4 mg) and microcrystalline cellulose powder (283 mg) were passed through mesh #35 and transferred to an appropriate traditional blender. Encapsulated ibuprofen (235 mg) and mannitol granules (385 mg) were added to the blender and mixed it for 5 minutes.

The flavor (10 mg), crosspovidone (70 mg), emulsifiers sodium lauryl sulfate (1 mg) and polysorbate 80 (1 mg) together were transferred to the blender and mixed it for 10 minutes.

Lubricant magnesium stearate (9 mg), talc (1.3 mg) and silicon dioxide (0.7 mg) were passed through # 60 mesh, and then transferred to the blender and blended further for 5 min

The resulting blend exhibited good granular flow. The blend was able to be compressed using conventional tablet press. Punch Size: 15.00 mm; Shape: Lozenge; Tablet weight: 1000 mg

Tablets had smooth mouth feel, tasted good and dissolved in the mouth in less than about 45 seconds.

### EXAMPLE 34

### Other Preparations of Ibuprofen

Ibuprofen Rapidmelt formulations analogous to that of Example 33 were prepared wherein the amount of ibuprofen per unit dosage form is changed to contain 50 mg or 100 mg of Ibuprofen. The amount of excipients may remain the same or adjusted slightly to accommodate formulation-related parameters such as flowability, compressibility, etc. The resulting tablets have essentially same characteristic and taste as Ibuprofen 200 mg. In both Examples 33 and 34, encapsulated Ibuprofen can be used for taste masking.

### EXAMPLE 35

### Preparation of Glucosamine HCl (750 mg) and Chondroitin Sulfate (100 mg) Chewmelts

Mono and diglyceride (74.14 g), distilled monoglycerides (172.70 g), lecithin (11.00 g) and acetylated mono-diglycerides (157.30 g) are added to the heating vessel. The mixture is heated up to 180° F. and stirred well till it melts.

Sigma mixer was preheated to temperature of 110° F.-130° F. and set to 60 RPM. The above molten mixture was transferred to the sigma mixer. Encapsulated glucosamine HCl (1137.40 g), chondroitin sulfate sodium (100.54 g), maltitol (355.30 g), flavors (22.22 g) and sweetener (4.84 g) were transferred to sigma mixer and mixed for 10 minutes. The mixture is cooled at ambient temperature. Then the mixture is subjected to size reduction by passing through multimill or any traditional mill, using a 4 mm screen then a 2 mm screen or mesh # 16 to get desired particle size granules. The granules were then transferred to an appropriate blender.

In the meantime, lubricants magnesium stearate (36.96 g), silicon dioxide (22.22 g), glidant talc (12.32 g), maltodextrin (66.44 g), solid flavors (24.42 g) and sweetener (3.74 g) were passed through # 35 mesh. After sifting the materials, the sifted materials were then transferred to the granules in blender and blended it together for 3 minutes.

The resulting blend exhibited good granular flow; the product was compressed using conventional tablet press. Punch Size: 0.6×1.0, Shape: Oval, Tablets were free from defects like pitting, chipping, & broken tablets.

Tablet weight: 2200 mg, Thickness of the tablet: 0.30″, Size of Tablet: 0.6″×1″, Friability: 0.2%, Water Activity: 0.24 Aw.

Dosage unit uniformity by weight variation:

(for Glucosamine HCl): Average: 102.1%, RSD: 2.3%,

(for Chondroitin Sulfate): Average: 102.5%, RSD: 1.6%,

Assay (for Glucosamine HCl): 103.0%, (for Chondroitin sulfate): 102.5%

Tablets were soft and easy bite, smooth feel, juicy, salivation creating and good in taste, thus the tablets have unique characteristics.

The shape, size, and weight of the tablet will vary to reflect the desired amount of Glucosamine HCl and Chondroitin Sulfate to be delivered.

### EXAMPLE 36

### Preparation of Glucosamine HCl (1000 mg) and Chondroitin Sulfate (100 mg) Chewmelts

Mono and diglyceride (40.00 g), distilled monoglycerides (40.00 g), lecithin (5.00 g) and acetylated mono-diglycerides (70.00 g) are added to the heating vessel. The mixture is heated up to 180° F. and stirred well till it melts.

Sigma mixer was preheated to temperature of 110° F.-130° F. and set to 60 RPM. The above molten mixture was transferred to the sigma mixer. Encapsulated glucosamine HCl (576.90 g), chondroitin sulfate sodium (37.50 g), sorbitol powder (101.30 g), citric acid (5.60 g) and flavors (11.00 g) were transferred to sigma mixer and mixed for 10 minutes. The mixture is cooled at ambient temperature. Then the mixture is subjected to size reduction by passing through multimill or any traditional mill, using a 4 mm screen then a 2 mm screen or mesh # 16 to get desired particle size granules. The granules were then transferred to an appropriate blender.

In the meantime, lubricants magnesium stearate (20.00 g), silicon dioxide (1.00 g), glidant talc (5.00 g), maltodextrin (59.70 g), solid flavors (15.00 g), sweetener (3.00 g) and color (0.50 g) were passed through # 35 mesh. After sifting the materials, the sifted materials were then transferred to the granules in blender and blended it together for 3 minutes.

The resulting blend exhibited good granular flow; the product was compressed using conventional tablet press. Punch Size: 0.6×1.0, Shape: Oval, tablet weight: 2667 mg, tablets were free from defects like pitting, chipping, & broken tablets.

Tablets were soft and easy bite, smooth feel, juicy, salivation creating and good in taste.

The shape, size, and weight of the tablet will vary to reflect the desired amount of Glucosamine HCl and Chondroitin Sulfate to be delivered.

### EXAMPLE 37

### Preparation of Glucosamine HCl (1500 mg) Chewmelts

Mono and diglyceride (20.00 g), distilled monoglycerides (20.00 g), lecithin (2.50 g) and acetylated mono-diglycerides (35.00 g) are added to the heating vessel. The mixture is heated up to 180° F. and stirred well till it melts.

Sigma mixer was preheated to temperature of 110° F.-130° F. and set to 60 RPM. The above molten mixture was transferred to the sigma mixer. Encapsulated glucosamine HCl (288.45 g), sorbitol powder (71.10 g), citric acid (2.80 g) and flavors (1.75 g) were transferred to sigma mixer and mixed for 10 minutes. The mixture is cooled at ambient temperature. Then the mixture is subjected to size reduction by passing through multimill or any traditional mill, using a 4 mm screen then a 2 mm screen or mesh # 16 to get desired particle size granules. The granules were then transferred to an appropriate blender.

In the meantime, lubricants magnesium stearate (10.00 g), silicon dioxide (5.00 g), glidant talc (2.50 g), maltodextrin (31.56 g), solid flavors (7.25 g) and sweetener (1.50 g) and color (0.50 g) were passed through # 35 mesh. After sifting the materials, the sifted materials were then transferred to the granules in blender and blended it together for 3 minutes.

The resulting blend exhibited good granular flow; the product was compressed using conventional tablet press. Punch Size: 0.6×1.0, Shape: Oval, tablet weight: 4000 mg, tablets were free from defects like pitting, chipping, & broken tablets.

Tablets were soft and easy bite, smooth feel, juicy, salivation creating and good in taste.

The shape, size, and weight of the tablet will vary to reflect the desired amount of Glucosamine HCl and Chondroitin Sulfate to be delivered.

### EXAMPLE 38

### Preparation of Caffeine 40 mg and Taurine 10 mg Chewmelts

Mono and diglycerides (40 g), distilled mono-diglycerides (75 g), acetylated mono-diglycerides (75 g) and lecithin (5 g) were added to the heating vessel. The mixture was heated up to 180° F. and stirred well till it melts.

Sigma mixer was preheated to temperature of 110° F.-130° F. and set to 60 RPM. The above molten mixture was transferred to the sigma mixer. Maltodextrin (295 g) and confectioners sugar (200 g) were passed through #35 mesh and were transferred to sigma mixer and mixed for 10 minutes. The mixture was cooled at ambient temperature.

Then the mixture was subjected to size reduction by passing through multimill or any traditional mill, using a 4 mm screen then a 2 mm screen or mesh # 16. The milled material was granular in nature. The granular milled material was then transferred to an appropriate blender.

In the meantime, lubricants magnesium stearate (10 g), silicon dioxide (10 g), encapsulated caffeine (117.6 g) and L-taurine (11.9 g), maltodextrin (122 g), flavors (18 g), citric acid (8 g), sweetener (1 g) and color agent (1.5 g) were passed through #35 screens. The sifted materials were then added over the granules in the blender and blended it together for 3 minutes.

The resulting blend exhibited good granular flow; the product was compressed using conventional tablet press.

Punch Size: 0.3500″×0.8500″, Shape: Rectangle, Tablet weight: 1000 mg, Tablets were free from defects like pitting, chipping, & broken tablets.

Tablets were soft and easy bite, smooth feel, juicy, salivation creating and good in taste.

### EXAMPLE 39

### Preparation of Extra Strength Antacid 1177 mg Chewmelts

Mono and diglycerides (330 g), distilled mono and diglycerides (90 g), acetylated mono diglycerides (225 g) and lecithin (22.50 g) were mixed in a heating vessel. The mixture was stirred and heated to a temperature up to 180° F. The mixture was maintained at 130° F. temperature till it melts completely.

Sigma mixer was preheated to temperature of 110° F.-130° F. and set to 60 RPM. The above molten mixture was transferred to the sigma mixer. Calcium carbonate (1412.40 g) was passed through #35 mesh. The sifted calcium carbonate, flavors (225 g) and sorbitol powder (594.60 g) transferred to sigma mixer and mixed for 10 minute. The mixture was cooled to ambient temperature.

Then the mixture was subjected to size reduction by passing through multimill or any traditional mill, using a 4 mm screen then a 2 mm screen or mesh # 16. The granules were then transferred to an appropriate blender.

In the meantime, lubricants magnesium stearate (15 g) and silicon dioxide (15 g), flavors (58.50 g), sweetener (7.50 g) and color agent (4.50 g) were passed through #35 screens. After sifting the materials, the sifted materials are then added over the granules in the blender and blended for 3 minutes.

The resulting blend exhibited good granular flow; the product was compressed using conventional tablet press. Punch Size: 0.6×1.0, Shape: Oval, Tablets were free from defects like pitting, chipping, & broken tablets.

Tablet Weight: 2500 mg, Thickness of the tablet: 0.30″, Size of Tablet: 0.6″×1″, Friability: 0.1%, Water Activity: 0.3 Aw, Acid Neutralizing Capacity (mEQ HCl/gram of chew tablet): 8.5%

Tablets were soft and easy bite, smooth feel, juicy, salivation creating and good in taste.

The blend was compressed to form tablets of weight of about 583 mg, or about 1167 mg, or about 1750 mg, or about 2334 mg, or about 2918 mg or about 3500 mg each. The 583 mg tablets each contained about 250 mg calcium per tablet. The 1167 mg tablets each contained about 500 mg calcium per tablet. The 1750 mg tablets each contained about 750 mg calcium per tablet. The 2334 mg tablets each contained about 1000 mg calcium per tablet. The 2918 mg tablets each contained about 1250 mg calcium per tablet. The 3500 mg tablets each contained about 1500 mg calcium per tablet.

The shape, size, and weight of the tablet will vary to reflect the desired amount of calcium carbonate to be delivered.

The preferred shape may be lozenge, oval or quadrisect for these supplements with such large quantities of calcium carbonate.

### EXAMPLE 40

### Preparation of Fiber 2 g Chewmelts

Mono and diglycerides (40.00 g) and distilled mono and diglycerides (80.00 g) were added to the Groen Kettle, heated it until the mixture melts. Then acetylated mono-diglycerides (75.00 g) and lecithin (5.00 g) were added to the Groen Kettle. The mixture was stirred and heated up to 180° F.

In the meantime, soluble fiber e.g., Fibersol-2 (640.50 g) and Citric acid (12.00 g) were sifted through # 35 mesh. Sigma mixer was preheated to temperature of 110° F.-130° F. and set to 60 RPM. The above molten mixture was transferred to the sigma mixer. The sifted maltodextrin and citric acid were then added to the molten mixture and mixed it for 10 minutes. The material was unloaded into plastic trays and cooled to room temperature.

Further, the Flexi Mill (Hammer Forward) was used for size reduction of the mass, the speed of auger of flexi mill was set at 8-10 RPM, speed of the blade was set at 1000-1200 RPM & 1.5 mm screens was used for size reduction. The material was unloaded in trays and kept in refrigerator for 2 hrs at 2-8° C.

The milled material and soluble fiber e.g. fibersol-2 (80.00 g), lubricant magnesium stearate (10.00 g), adsorbent silicon dioxide (10.00 g), flavors (30.00 g), citric acid (15.00 g), sucralose (1.00 g) and color (1.50 g) were added into PADC blender and mixed it for 10 min at 7 RPM. The resulting blend exhibited good granular flow.

Tablet Press compression machine was set with standards of oval shaped punches, punch dimension was 0.6 inch×1.0 inch. The blend was compressed to form tablets of weight of about 1375 mg, or about 2063 mg, or about 4125 mg each. The 1375 mg tablets each contained about 1 g fiber per tablet. The 2063 mg tablets each contained about 1.5 g fiber per tablet. The 4125 mg tablets each contained about 3.0 g calcium per tablet. Tablets were free from defects like pitting, chipping, & broken tablets. The shape, size, and weight of the tablet will vary to reflect the desired amount of fiber to be delivered.

The preferred shape may oval or quadrisect for these supplements with such large quantities of fiber.

Tablets were soft and easy bite, smooth feel, juicy, salivation creating and good in taste.

### EXAMPLE 41

### Preparation of Fiber 1.5 g and Phytosterols (2.0 g) Chewmelts

Mono and diglyceride (40.00 g), distilled monoglycerides (80.00 g), lecithin (5.00 g) and acetylated mono-diglycerides (75.00) are added to the heating vessel. The mixture is heated up to 180° F. and stirred well till it melts.

Sigma mixer was preheated to temperature of 110° F.-130° F. and set to 60 RPM. The above molten mixture was transferred to the sigma mixer. Soluble fiber e.g. Fibersol-2 (228.12) and phytosterols (412.39 g), and citric acid (12.00 g) were transferred to sigma mixer and mixed for 10 minutes. The mixture is cooled at ambient temperature. Then the mixture is subjected to size reduction by passing through multimill or any traditional mill, using a 4 mm screen then a 2 mm screen or mesh # 16 to get desired particle size granules. The granules were then transferred to an appropriate blender.

In the meantime, lubricants magnesium stearate (10.00 g), silicon dioxide (10.00 g), maltodextrin (87.00 g), citric acid (8.00 g), solid flavors (30.00 g), sweetener (1.00 g) and color (1.50 g) were passed through # 35 mesh. After sifting the materials, the sifted materials were then transferred to the granules in blender and blended it together for 3 minutes.

The resulting blend exhibited good granular flow; the product was compressed using conventional tablet press. Punch Size: 0.6×1.0, Shape: Oval, tablet weight: 4850 mg, tablets were free from defects like pitting, chipping, & broken tablets.

### EXAMPLE 42

### Preparation of Fiber 1.75 g, Caffeine 2.5 mg and Green Tea 22.5 mg Chewmelts

Mono and diglycerides (90 g), distilled mono and diglycerides (180 g), acetylated mono-diglycerides (168.75 g), and Lecithin (11.25 g) were added to the heating vessel. The mixture was heated up to 180° F. and stirred well till it melts.

Sigma mixer was preheated to temperature of 110° F.-130° F. and set to 60 RPM. The above molten mixture was transferred to the sigma mixer. Then soluble fiber e.g. fibersol-2 (1800 g) was added to the molten mixture under continuous stirring and then mixed it for 3 minutes. Then cooled it to room temperature. Then the mixture was subjected to size reduction by passing through multimill or any traditional mill, using a 4 mm screen then a 2 mm screen or mesh # 20. The resulting granules exhibited good granular flow. The granules were transferred to the blender.

Then Maltodextrin (1800 g), Green Tea Flavor (22.50 g) and color (3.38 g) were sifted through mesh #35 and transferred it to the double cone blender. Encapsulated caffeine 40% (31.28 g) was added to the double cone blender and blended it for 5 min.

The lubricants magnesium stearate (22.50 g), silicon dioxide (22.50 g), solid flavors (67.50 g) and sweetener (2.25 g) were passed through #35 screens and were added to the granules in the blender and blended for additional 3 minutes.

The resulting blend exhibited good granular flow; the product was compressed using conventional tablet press. Punch Size: 0.6×1.0, Shape: Oval, Tablet weight: 2250 mg, Tablets were free from defects like pitting, chipping, & broken tablets.

Tablets were soft and easy bite, smooth feel, juicy, salivation creating and good in taste.

### EXAMPLE 43

### Preparation of Fiber 2 gm and Calcium 250 mg and Vitamin D3K 100 iu Chewmelts

Mono and diglycerides (120 g), distilled mono and diglycerides (240 g), acetylated mono-diglycerides (225 g), and Lecithin (15 g) were added to the heating vessel. The mixture was heated up to 180° F. and stirred well till it melts.

The molten mixture was added to the soluble fiber e.g. fibersol-2 (1957.47 gm) under continuous stirring and then mixed it for 3 minutes. Then cooled it to room temperature. Then the mixture was subjected to size reduction by passing through multimill or any traditional mill, using a 4 mm screen then a 2 mm screen or mesh # 20. The resulting granules exhibited good granular flow. The granules were transferred to the blender.

The lubricants magnesium stearate (30 g), silicon dioxide (15 g), vitamin D3 and vitamin K premix (68.75 g), calcium carbonate (252.6 g), soluble fiber e.g. fibersol-2 (43.00 g), solid flavors (54 g), sweetener (3 g) and color agent (4.5 g) were passed through #35 screens and were added to the granules in the blender and blended for 3 minutes.

The resulting blend exhibited good granular flow; the product was compressed using conventional tablet press. Punch Size: 0.6×1.0, Shape: Oval, Tablets were free from defects like pitting, chipping, & broken tablets.

Tablet weight: 3000 mg, Thickness of the tablet: 0.30″, Size of Tablet: 0.6″×1″, Friability: 0.1%

Tablets were soft and easy bite, smooth feel, juicy, salivation creating and good in taste.

### EXAMPLE 44

### Preparation of Zinc 10.5 mg Chewmelts

Spray dried menthol (15 g), milled zinc acetate dehydrate (27.5 g), zinc gluconate (19.1 g), HPMC (5 g), Maltodextrin M180 (240 g), Bakers Special Granular Sugar (346 g) & sweetner (3.75 g) were sifted through mesh # 35. Transferred all the materials to the Hobart mixture and mixed it for 3 minutes.

Transfer mono and diglycerides (40 g), distilled mono and diglycerides (60 g), acetylated mono-diglycerides (70 g), polyethylene glycol (20 g) and lecithin (5 g) to the heating vessel. The mixture was heated up to 180° F. and stirred well till it melts.

Then transfer the molten mixture to the preheated sigma mixer under continuous stirring. Then add eucalyptus oil (1.1 g) and other flavors (20.5 g) to the sigma mixture under continuous stirring and then mixed for 7-10 minutes. The material was cooled to ambient temperature.

Then the mixture was subjected to size reduction by passing through multimill or any traditional mill, using a 4 mm screen then a 2 mm screen or mesh # 20. The resulting granules exhibited good granular flow. The granules were transferred to the blender.

The lubricants magnesium stearate (10 g), silicon dioxide (10 g), maltodextrin M 180 (104.2 g), flavors (15 g), sweetener (1.25 g) and color agent (1.50 g) were passed through # 35 screens and then added to the granular material in the blender and blended for 3 minutes.

The resulting blend exhibited good granular flow; the product was compressed using conventional tablet press. Punch Size: 0.6×1.0, Shape: Oval, Tablets were free from defects like pitting, chipping, & broken tablets.

Tablet weight: 3000 mg, Thickness of the tablet: 0.30″, Size of Tablet: 0.6″×1″, Friability: 0.1%

Tablets were soft and easy bite, smooth feel, juicy, salivation creating and good in taste.

### EXAMPLE 45

### Preparation of Calcium 500 mg, VitD3 100 IU K40 mcg Chewmelts

Mono and diglycerides (300.82 g), and distillated mono diglycerides (82.04 g), acetylated mono-diglycerides (300.82 g) and lecithin (20.51 g) were added to the Groen Kettle. The mixture was stirred and heated up to 180° F. to melt.

The molten mixture was transferred to preheated sigma mixer; calcium carbonate (1287.50 g) was added to it and mixed it for 2 min. Then non fat dry milk grade A (205.10 g) and sorbitol powder (542.02 g) were added to sigma mixer and mixed it for 15 min. The mass was cooled to the room temperature.

Further, the Flexi Mill (Hammer Forward) was used for size reduction of the mass, the speed of auger of flexi mill was set at 8-10 RPM, speed of the blade was set at 1000-1200 RPM & 1.5 mm screens was used for size reduction. The material was unloaded in totes and kept in refrigerator for 2 hrs at 2-8° C.

The milled materials and lubricants magnesium stearate (13.67 g) and silicon dioxide (13.67 g), sucralose (6.84 g), flavors (53.32 g), and color (4.10 g) were add into PADC blender and mixed it for 10 min at 7 RPM. The resulting blend exhibited good granular flow.

Tablet Press compression machine was set with standards of oval shaped punches, punch dimension was 0.6 inch×1.0 inch, the blend was compressed. Tablets were free from defects like pitting, chipping, & broken tablets.

Tablets were soft and easy bite, smooth feel, juicy, salivation creating and good in taste.

### EXAMPLE 46

### Preparation of Balsalazide 2.2 g Chewmelts

Mono and diglyceride (20.00 g), distilled monoglycerides (20.00 g), lecithin (2.50 g) and acetylated mono-diglycerides (35.00 g) are added to the heating vessel. The mixture is heated up to 180° F. and stirred well till it melts.

Sigma mixer was preheated to temperature of 110° F.-130° F. and set to 60 RPM. The above molten mixture was transferred to the sigma mixer. Balsalazide (275 g), sorbitol (84.55 g), citric acid (2.80 g) and flavors (1.75 g) were transferred to sigma mixer and mixed for 10 minutes. The mixture is cooled at ambient temperature. Then the mixture is subjected to size reduction by passing through multimill or any traditional mill, using a 4 mm screen then a 2 mm screen or mesh # 16 to get desired particle size granules. The granules were then transferred to an appropriate blender.

In the meantime, lubricants magnesium stearate (10.00 g), silicon dioxide (5.00 g), glidant talc (2.50 g), maltodextrin (31.65 g), solid flavors (7.25 g), sweetener (1.50 g) and color (0.50 g) were passed through # 35 mesh. After sifting the materials, the sifted materials were then transferred to the granules in blender and blended it together for 3 minutes.

The resulting blend exhibited good granular flow; the product was compressed using conventional tablet press. Tablet weight: 4000 mg, Tablets were free from defects like pitting, chipping, & broken tablets. The preferred shape may quadrisect with such large quantities of active ingredient.

### EXAMPLE 47

### Preparation of Oral Anesthetic and Oral Analgesic Benzocaine 6 mg Flashbeads—Grape Flavor

Cocoa butter (480.02 g), polyethylene glycol (120.00 g) and sorbitan monostearate (80.00 g) were added to the groen kettle and heated it to 140° F. Towards the end of melting, sodium lauryl sulphate (2.00 g) and polysorbate 80 (6.00 g) were added to it and mixed using a stainless steel spatula. The temperature was maintained at approximately 115° F.-140° F.

Sugar (1690.47 g) and benzocaine (49.44 g) were sifted through # 25 mesh. After sieving, the material was added in a suitable mixer under low shear conditions e.g. hobart mixer and mixed for 5 minutes. The mixture was transferred to the double cone blender. Grape flavor (12.00 g), sucralose (20.00 g), and crosspovidone (120.00 g) were sifted through mesh # 25 and the sieved materials were added to double cone blender. The materials were blended together for 5 minutes at 7-10 RPM.

The sigma mixer was set at 15 RPM. The steam valve was opened and preheated the mixer bowl to approximately 110° F.-120° F. The above molten mixture was added to the sigma mixer. It was mixed for 2 minutes in forward direction. The blend from double cone blender was transferred to the sigma mixer at temperature approx. 115° F. and mixed for 3 minutes in forward direction. Granulated Sugar (1400.06 g) was added to the sigma mixer and mixed it for 4 minutes in forward direction. The material should be slightly lumpy soft granular in nature.

The granulated material was extruded through twin screw cone extruder using with 1.0 mm cone mesh and spheronized using a spheronizer having a 3.25 mm chequered plate at 123-124 RPM for 20 seconds. The beads were spread uniformly in trays to cool down to room temperature (63° F. -73° F.). Flashbeads were white, spherical to near spherical and free flowing.

Bulk density of benzocaine flashbeads: 0.7 g/mL

Particle size: % retained on # 16 is 6.1%, % retained on # 25 is 90.6%, % retained on # 35 is 3.0%, % retained on # 40 is 0.1% and passed through # 40 is 0.3%

Flashbeads melted within less than 6 seconds of being placed in the mouth of mammal.

### EXAMPLE 48

### Preparation of Guaifenesin 100 mg Flashbeads—Grape Flavor

Cocoa butter (60.00 g), polyethylene glycol (15.00 g) and Sorbitan monostearate (10.00 g) were added to the groen kettle and heated it to 140° F. Towards the end of melting, sodium lauryl sulphate (0.30 g) and polysorbate 80 (0.80 g) were added to it and mixed it using a stainless steel spatula. The temperature was maintained at approximately 115° F.-140° F.

Sugar (211.30 g) was sifted through # 25 mesh. Encapsulated guaifenesin 30% (333.33 g) and sifted sugar were added in a suitable mixer under low shear conditions e.g. Hobart mixer and mixed for 5 minutes. The mixture was transferred to the double cone blender. Grape flavor (1.50 g), sucralose (2.50 g), and crosspovidone (15.00 g) were sifted through mesh # 25 and added to double cone blender. The two mixtures were blended together for 5 minutes at 7-10 RPM.

The Sigma mixer was set at 15 RPM. The steam valve was opened and preheated the sigma mixer bowl to approximately 110° F.-120° F. The above molten mixture was added to the sigma mixer. It was mixed for 2 minutes in forward direction. The blend from double cone blender was transferred to the sigma mixer at temperature approx. 115° F. and mixed for 3 minutes in forward direction. Granulated sugar (175.00 g) was added to the sigma mixer and mixed for 4 minutes in forward direction. The material should be slightly lumpy soft granular in nature.

The granulated material was extruded through twin screw cone extruder using with 1.0 mm cone mesh and spheronized using a spheronizer having a 3.25 mm chequered plate at 123-124 RPM for 20 seconds. The beads were spread uniformly in trays to cool down to room temperature (63° F.-73° F.). Flashbeads were white, spherical to near spherical and free flowing.

Flashbeads melted within less than 5 seconds of being placed in the mouth of mammal.

### EXAMPLE 49

### Preparation of Placebo Flashbeads—Grape Flavor

Cocoa butter (6.00 kg), polyethylene glycol (1.50 kg) and sorbitan monostearate (1.50 kg) were added to the groen kettle and heated it to 140° F. Towards the end of melting, sodium lauryl sulphate (0.03 kg) and polysorbate-80 (0.08 kg) were added to it and mixed it using a stainless steel spatula. The temperature was maintained at approximately 115° F.-140° F.

Sugar (21.24 kg) was sifted through # 25 mesh & transferred to the double cone blender. Grape flavor (0.15 kg), sucralose (0.25 kg), and crosspovidone (1.50 kg) were sifted through mesh # 25 and added to double cone blender. The materials were blended together for 5 minutes at 7-10 RPM.

The Sigma mixer was set at 15 RPM. The steam valve was opened and preheated the sigma mixer bowl to approximately 110° F.-120° F. The above molten mixture was added to the sigma mixer. It was mixed for 2 minutes in forward direction. The blend from double cone blender was transferred to the sigma mixer at temperature approx. 115° F. and mixed for 3 minutes in forward direction. Granulated sugar (17.50 kg) was added to the sigma mixer and mixed for 4 minutes in forward direction. The material should be slightly lumpy soft granular in nature.

The granulated material was extruded through twin screw cone extruder using with 1.0 mm cone mesh and spheronized using a spheronizer having a 3.25 mm chequered plate at 123-124 RPM for 20 seconds. The beads were spread uniformly in trays to cool down to room temperature (63° F.-73° F.). Flashbeads were white to off-white, spherical to near spherical and free flowing.

Flashbeads melted within less than 5 seconds of being placed in the mouth of mammal.

Bulk density 0.6 g/mL, particle size: retained on # 16 mesh: 2.83%, retained on # 25 mesh: 72.36%, retained on # 35 mesh: 16.69%, retained on # 40 mesh: 2.22%, retained on # 60 mesh: 4.95% and passed through # 60 mesh: 0.95%

### EXAMPLE 50

### Blending Process of Placebo Flashbeads—Grape Flavor and Encapsulated Guaifenesin Beads

The placebo flashbeads—grape flavor (744 g) of example 49 and encapsulated guaifenesin beads 30% (178 g) were transferred to an appropriate blender and blended it for 10 minutes at 7 RPM.

Beads of encapsulated Guaifenesin having bulk density 0.70 g/mL,

Particle size: retained above # 16 is 0.1%, above # 20 is 30.2%, above # 25 is 65.4%, above # 35 is 4.2%

### EXAMPLE 51

### Packaging of Blend

The blended mixture of placebo flashbeads—grape flavor and encapsulated guaifenesin beads were filled in the stick packs to provide 50 mg and 100 mg guaifenesin per unit dosage form.

When the packaged mixture of placebo flashbeads—grape flavor and encapsulated guaifenesin beads is placed in mouth of a user, Flashbeads melted within less than 5 seconds of being placed in the mouth of mammal and the unique novel combination allows for fast swallowing of the encapsulated drug beads.

The stability study showed that the product was stable in the stick packs and it maintains the characteristics.

### EXAMPLE 52

### Preparation of Acetaminophen 80 mg Flashbeads—Grape Flavor

Flashbeads formulation were prepared as described in Example 47 with the following changes: sorbitan monostearate (80.00 g), cocoa butter (480.00 g), polyethylene glycol (120.00 g), sodium lauryl sulphate (2.00 g) and polysorbate 80 (6.00 g), sugar (1033.60 g) and encapsulated acetaminophen 91% (706.04), grape flavor (12.00 g), sucralose (20.00 g), and crosspovidone (120 g), granulated sugar (1400 g). Flashbeads were white to off-white, spherical to near spherical and free flowing.

Flashbeads melted within 6-8 seconds of being placed in the mouth of mammal.

### EXAMPLE 53

### Preparation of Acetaminophen Flashbeads—Grape Flavor

TheA placebo flashbeads—grape flavor (750 g) of example 49 and encapsulated acetaminophen 91% (250 g) were transferred to the double cone blender and blended it for 10 minutes at 7 RPM. The blended mixture of placebo flashbeads—grape flavor and encapsulated acetaminophen 91% was filled in the stick packs to provide 80 mg and 160 mg acetaminophen per unit dosage form. The placebo flashbeads—grape flavor and encapsulated acetaminophen 91% can optionally be filled from two hoppers in the packet.

Encapsulated Acetaminophen 91%: Bulk density: 0.49 g/ml, Particle size: % retained on # 40 is 28% and on # 80 is 70%

The stability study showed that the product was stable in the stick packs and it maintains the characteristics.

### EXAMPLE 54

### Preparation of Guaifenesin 100 mg and Dextromethorphen HBr 15 mg Flashbeads—Grape Flavor

Flashbeads formulation were prepared as described in Example 47 wherein the active ingredient added were taste masked acetaminophen and taste masked dextromethorphan and the amount of active ingredients per unit dosage form was 100 mg and 15 mg respectively. The ratio of excipients may remain the same or adjusted slightly to accommodate formulation-related parameters.

Flashbeads melted within less than 5 seconds in mouth.

### EXAMPLE 55

### Preparation of Guaifenesin 100 mg and Dextromethorphen HBr 15 mg Flashbeads—Grape Flavor

The placebo flashbeads—grape flavor (800 g) of example 49 and encapsulated guaifenesin 30% (200 g) and encapsulated dextromethorphan HBr 1.5% (30 g) were transferred to the double cone blender and blended it for 10 minutes at 7 RPM. The blended mixture of placebo flashbeads—grape flavor and encapsulated guaifenesin 30% with dextromethorphan HBr 1.5% was filled in the stick packs to provide guaifenesin 100 mg and dextromethorphen HBr 15 mg per unit dosage form. The placebo flashbeads—grape flavor, encapsulated guaifenesin 30% and encapsulated dextromethorphan HBr 1.5% can optionally be filled from two or more hoppers in the packet.

### EXAMPLE 56

### Preparation of Doxycycline Flashbeads—Grape Flavor

Flashbeads formulation were prepared as described in Example 47 wherein the active ingredient added was taste masked doxycycline or doxycycline calcium and the amount of active ingredients per unit dosage form was 25 mg and 50 mg respectively. The ratio of excipients may remain the same or adjusted slightly to accommodate formulation-related parameters. Flashbeads melted within less than 5 seconds of being placed in the mouth of mammal.

### EXAMPLE 57

### Preparation of Doxycycline Flashbeads—Grape Flavor

The placebo flashbeads—grape flavor (750 g) of example 49 and taste masked doxycycline (250 g) or taste masked doxycycline calcium (250 g) were transferred to the double cone blender and blended it for 10 minutes at 7 RPM. The blended mixture of placebo flashbeads—grape flavor and taste masked doxycycline or taste masked doxycycline calcium was filled in the stick packs to provide doxycycline 25 mg and 50 mg per unit dosage form. The placebo flashbeads—grape flavor and taste masked doxycycline can optionally be filled from two hoppers in the packet.

### EXAMPLE 58

### Preparation of Ciprofloxacin Flashbeads—Grape Flavor

Flashbeads formulation were prepared as described in Example 47 wherein the active ingredient added was taste masked ciprofloxacin and the amount of active ingredients per unit dosage form was 250 mg. The ratio of excipients may remain the same or adjusted slightly to accommodate formulation-related parameters. Flashbeads melted within less than 5 seconds of being placed in the mouth of mammal.

### EXAMPLE 59

### Preparation of Ciprofloxacin Flashbeads—Grape Flavor

The placebo flashbeads—grape flavor (750 g) of example 49 and taste masked ciprofloxacin (250 g) were transferred to the double cone blender and blended it for 10 minutes at 7 RPM. The blended mixture of placebo flashbeads—grape flavor and taste masked ciprofloxacin was filled in the stick packs to provide ciprofloxacin 250 mg and 500 mg per unit dosage form. The placebo flashbeads—grape flavor and taste masked ciprofloxacin can optionally be filled from two hoppers in the packet.

### EXAMPLE 60

### Preparation of Amoxicillin and Clavulanate Potassium Flashbeads—Grape Flavor

Flashbeads formulation were prepared as described in Example 47 wherein the active ingredient added were taste masked amoxicillin and clavulanic acid as the potassium salt and the amount of active ingredients per unit dosage form was 125/32 mg, 200/29 mg, 250/63 mg, and 400/57 mg. The ratio of excipients may remain the same or adjusted slightly to accommodate formulation-related parameters. Flashbeads melted within less than 5 seconds of being placed in the mouth of mammal.

### EXAMPLE 61

### Preparation of Amoxicillin and Clavulanate Potassium Flashbeads—Grape Flavor

The placebo flashbeads—grape flavor (750 g) of example 49 and taste masked amoxicillin (152 g) and clavulanate potassium (32 g) were transferred to the double cone blender and blended it for 10 minutes at 7 RPM. The blended mixture of placebo flashbeads—grape flavor and taste masked amoxicillin and clavulanate potassium was filled in the stick packs to provide amoxicillin and clavulanate potassium 125/32 mg, 200/29 mg, 250/63 mg, and 400/57 mg per unit dosage form. The placebo flashbeads—grape flavor, taste masked amoxicillin and clavulanate potassium can optionally be filled from two or more hoppers in the packet.

### EXAMPLE 62

### Preparation of Risperidone Flashbeads—Grape Flavor

Flashbeads formulation were prepared as described in Example 47 wherein the active ingredient added was taste masked risperidone and the amount of active ingredients per unit dosage form was 0.5 mg and 1 mg. The ratio of excipients may remain the same or adjusted slightly to accommodate formulation-related parameters. Flashbeads melted within less than 5 seconds of being placed in the mouth of mammal.

### Example 63

### Preparation of Risperidone Flashbeads—Grape Flavor

The placebo flashbeads—grape flavor (900 g) of example 49 and taste masked risperidone (100 g) was transferred to the double cone blender and blended it for 10 minutes at 7 RPM. The blended mixture of placebo flashbeads—grape flavor and taste masked risperidone was filled in the stick packs to provide risperidone 0.5 mg and 1 mg per unit dosage form. The placebo flashbeads—grape flavor and taste masked risperidone can optionally be filled from two hoppers in the packet.

### EXAMPLE 64

### Preparation of Sumatriptan Succinate Flashbeads—Grape Flavor

Flashbeads formulation were prepared as described in Example 47 wherein the active ingredient added was encapsulated sumatriptan 25% and the amount of active ingredients per unit dosage form was 25 mg, 50 mg and 100 mg. The ratio of excipients may remain the same or adjusted slightly to accommodate formulation-related parameters. Flashbeads melted within less than 5 seconds of being placed in the mouth of mammal.

### EXAMPLE 65

### Preparation of Sumatriptan Succinate Flashbeads—Grape Flavor

The placebo flashbeads—grape flavor (750 g) of example 49 and encapsulated sumatriptan 25% (250 g) was transferred to the double cone blender and blended it for 10 minutes at 7 RPM. The blended mixture of placebo flashbeads—grape flavor and encapsulated sumatriptan 25% was filled in the stick packs to provide sumatriptan succinate 25 mg, 50 mg and 100 mg per unit dosage form. The placebo flashbeads—grape flavor and encapsulated sumatriptan 25% can optionally be filled from two hoppers in the packet.

### EXAMPLE 66

### Preparation of Olanzapine Flashbeads 10 mg—Grape Flavor

Flashbeads formulation were prepared as described in Example 47 wherein the active ingredient added was olanzapine and the amount of active ingredients per unit dosage form was 5 mg, 10 mg, 15 mg and 20 mg. The ratio of excipients may remain the same or adjusted slightly to accommodate formulation-related parameters. Flashbeads melted within less than 5 seconds of being placed in the mouth of mammal.

### EXAMPLE 67

### Preparation of Olanzapine Flashbeads 10 mg—Grape Flavor

The placebo flashbeads—grape flavor (750 g) of example 49 and olanzapine (250 g) was transferred to the double cone blender and blended it for 10 minutes at 7 RPM. The blended mixture of placebo flashbeads—grape flavor and olanzapine was filled in the stick packs to provide olanzapine 5 mg, 10 mg, 15 mg and 20 mg per unit dosage form. The placebo flashbeads—grape flavor and olanzapine can optionally be filled from two hoppers in the packet.

### EXAMPLE 68

### Preparation of Diphenhydramine HCl 25 mg Flashbeads—Black Cherry Flavor

Cocoa butter (60.00 g) and polyethylene glycol (15.00 g) were added to the heating vessel, heated it and temperature was maintained below approximately 115° F.-140° F. Towards end of melting polysorbate 80 (0.75 g), sodium lauryl sulphate (0.25 g) and sorbitian monostearate (15.00 g) were added to it and mixed it with spatula.

Encapsulated diphenhydramine 9% (277.78 g), Sugar (201.30 g), sucralose (2.50 g) and crosspovidone (15.00 g) were sifted through #25 mesh and transferred into blender and blended it for 2 minutes. Black cherry flavor (500.00 g) and granulated sugar (175.00 g) were added to the blender and blended further for 2 minutes.

The above blended mixture was added slowly to the molten mixture in 15 minutes and mixed well with spatula. This granular material was cooled to room temperature (63° F.-73° F.) for 20 minutes. The granulated material was extruded through an extruder using a 0.8 mm screen and 100 RPM for 7 minute to make long cylindrical extrudate. The extrudate was cooled for 15 minutes in a refrigerator. The extrudate was spheronized using a spheronizer having a 3.25 mm pitch spheronization plate at 900 RPM-1200 RPM for 3 minutes. The beads were spread uniformly in trays to cool down to room temperature (63° F.-73° F.). Flashbeads were spherical to near spherical and free flowing. Flashbeads melted within 5-6 seconds of being placed in the mouth of a mammal.

### EXAMPLE 69

### Preparation of Diphenhydramine HCl 25 mg Flashbeads—Black Cherry Flavor

The placebo flashbeads—black cherry flavor formulation was prepared as described in example 49 except the flavor used was Black Cherry Flavor. The placebo flashbeads—black cherry flavor (750 g) and encapsulated diphenhydramine 9% (250 g) were transferred to the double cone blender and blended it for 10 minutes at 7 RPM. The blended mixture of placebo Flashbeads—Black Cherry Flavor and Encapsulated diphenhydramine 9% was filled in the stick packs to provide Diphenhydramine HCl 25 mg per unit dosage form. The placebo flashbeads—black cherry flavor and encapsulated diphenhydramine 9% can optionally be filled from two hoppers in the packet.

Encapsulated Diphenhydramine HCl beads 10%

Bulk density: 0.75 g/ml

Particle size: % retained on # 40 is 3.9%, # 60 is 13.0%, # 80 is 21.8% and # 100 is 9.6%

### EXAMPLE 70

### Preparation of Ibuprofen 100 mg Flashbeads—Grape Flavor

Cocoa butter (60.00 g) and polyethylene glycol (15.00 g) were taken in the heating vessel and heated it. The temperature was maintained to approximately 115° F.-140° F. Towards end of melting, polysorbate 80 (0.75 g), sodium lauryl sulphate (0.25 g) and sorbitian monostearate (15.00 g) were added and mixed it well with spatula.

Encapsulated Ibuprofen 10% (1000 g), Sugar (201.30 g), Sucralose (2.50 g) and crosspovidone (15.00 g) were sifted through #25 mesh and transferred it into the blender and blended it for 2 minutes. Grape flavor (500.00 g) and granulated sugar (175.00 g) were added to the blender and blended further for additional 2 minutes.

The blended mixture was slowly added to the molten mixture in 15 minutes and mixed well by using spatula. This granular material was cooled to room temperature (63° F.-73° F.) for 20 mins. The granulated material was extruded through an extruder using a 0.8 mm screen and 98.5 RPM for 7 min to make round, long, threaded, plain extrudate. The extrudate were cooled for 15 minutes in a refrigerator. The mass was spheronized using a spheronizer having a 3.25 mm pixture spheronization plate at 900 RPM-1200 RPM for 3 minutes. The beads were spread uniformly in trays to cool down to room temperature (63° F.-73° F.). Flashbeads were white, spherical to near spherical and free flowing. Flashbeads were spherical to near spherical and free flowing. Flashbeads melted within 5-6 seconds of being placed in the mouth of a mammal.

### EXAMPLE 71

### Preparation of Ibuprofen 100 mg Flashbeads—Black Cherry Flavor

The placebo flashbeads—grape flavor (750 g) and encapsulated ibuprofen 10% (250 g) were transferred to the double cone blender and blended it for 10 minutes at 7 RPM. The blended mixture of placebo flashbeads—grape flavor and encapsulated ibuprofen 10% was filled in the stick packs to provide Ibuprofen 100 mg per unit dosage form The placebo flashbeads—black cherry flavor and encapsulated ibuprofen 10% can optionally be filled from two hoppers in the packet.

### EXAMPLE 72

### Preparation of Zinc 11 mg Flashbeads—Cherry Flavor

Natural black cherry flavor (0.5 kg), sodium lauryl sulfate (0.025 kg), mannitol powder (2.500 kg), povidone (0.05 kg), sorbitan monostearate (0.125 kg), sucralose (0.075 kg) & acesulfame K (0.063 kg) powder were sifted through # 24 screen. Zinc acetate dihydrate (0.820 kg) and zinc gluconate (0.585 kg) were added to vibratory sifter and pass through # 24 screen.

Crosspovidone premix was prepared by mixing crospovidone, sodium lauryl sulfate and sodium starch glycolate together in sigma mixer bowl. The sigma mixer was started at speed 30 RPM and polysorbate 80 was added slowly to the sigma mixer bowl and mixed for 10 minutes at 30 RPM after completing the addition.

Crosspovidone premix (1.170 kg) and crospovidone (1.143 kg) were added to vibratory sifter and pass through # 24 screen. and added to the above sifted materials.

The lubricants talc (0.05 kg), magnesium stearate (0.15 kg) and silicon dioxide (0.025 kg) were sifted through # 24 mesh. Mannitol Granules (12.218 kg) was sifted through # 24 screen.

Half of sifted mannitol granules, microcrystalline cellulose powder (5.5 kg), above sifted material, remaining half of mannitol granules were added to 75 L double cone blender and blended for 20 min at 8 RPM. After completion of 20 minutes of initial blending sifted lubricants were added into 75 L Double cone Blender and mixed for 5 minutes at 8 RPM. The resulting blend exhibited good granular flow. The blend can be filled with Flashbeads in a packet.

### EXAMPLE 73

### Preparation of Acetaminophen Flashbeads—Cherry Flavor

Mannitol granules (152 g) was sifted through # 25 mesh. Grape flavor (4.9 g) and sweetener (2.8 g) were added to it and kept this mixture aside as premix 1.

Crospovidone (49 g), sodium lauryl sulphate (0.7 g) and sodium starch glycolate (14 g) were mixed together. polysorbate 80 (1.05 g) was slowly added to this mixture which acts as emulsifier. After completely adding the polysorbate 80, mixed it for 10 minutes. Sieved the mixture from # 35 mesh and kept this mixture aside as premix 2.

Then the lubricants magnesium stearate (4.20 g) and silicon dioxide (1.4 g) were sifted from # 25 mesh. Mannitol granules (152.71 g), microcrystalline cellulose (140 g) and color agents (1.4 g) were sifted through # 25 mesh. This mixture was transferred to the appropriate blender and the encapsulated Acetaminophen 91% (175.84 g) was added to it and blended for 20 minutes.

Premix 2 was transferred to the blender and blended for another 10 minutes. Then the flavor premix 1 was added to the blender and blended for another 15 minutes. Then the lubricants magnesium stearate (4.20 g) and silicon dioxide were added to the blender and blended for an additional 5 minutes. The resulting blend exhibited good granular flow. The blend can be filled with flashbeads in a packet.

### EXAMPLE 74

### Preparation of Placebo Flashbeads—Orange Flavor

Diluent sugar (201.30 g), sweetening agent sucralose (2.50 g) and crosspovidone (15.00 g) were passed through #25 mesh, transferred it in to blender and blended it for 2 mins. Flavor (12.70 g) and diluent granulated sugar (175.00 g) were sifted through # 25 mesh and added to the blender and blend it for additional 2 minutes.

Cocoa butter (60.00 g) and polyethylene glycol granular (15.00 g) were added to the heating vessel and heated it. The temperature was maintained below approximately 115° F.-140° F. Towards end of melting polysorbate-80 (0.75 g), sodium lauryl sulphate (0.25 g) and sorbitian monostearate (15.00 g) were added and mixed it using a stainless steel spatula.

The above blended mixture was slowly added to above molten mixture in 15 minutes and mixed well by using spatula. This granular material cooled to room temperature (63° F.-73° F.) for 20 minutes. The granulated material was extruded through an extruder using a 0.8 mm screen and 100 RPM for 7 minutes to make round, long, threaded, plain extrudate. The extrudate were kept in a refrigerator for 15 minutes. The mass was spheronized using a spheronizer having a 3.25 mm chequered plate at 900 RPM-1200 RPM for 3 mins. Silicon dioxide (2.50 g) was sifted through #25 mesh and added to the finished product to avoid the static. Flashbeads were spherical to near spherical and free flowing. Flashbeads melted within less than 5 seconds of being placed in the mouth of mammal.

Placebo Flashbeads formulations analogous to Examples 49 and 74 were prepared wherein the flavor was changed to give different flavored placebo flashbeads. The flavored placebo flashbeads were used in combination with drug beads, granules, powder or crystals. Drug beads can be taste masked or encapsulated drug beads.

Flashbeads can be mixed with other pharmaceutically acceptable excipients to be formulated as dry syrups, suspensions, sachets or any other suitable oral dosage forms.

The taste masked drug beads can be mixed with flavored placebo flashbeads and with other pharmaceutically acceptable excipients to be formulated as dry syrups, suspensions, sachets or any other suitable oral dosage forms.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A rapid-melt pharmaceutical composition comprising:
a binder;
a super emulsifier;
a diluent/bulking material; and
an active ingredient.

2. The composition of claim 1, wherein the super emulsifier has a hydrophilic-lipophilic balance of greater than about 10.

3. The composition of claim 1, wherein the super emulsifier is selected from the group consisting of polyethoxylated fatty acids, polyethylene glycol (PEG) fatty acid diesters, PEG glycerol fatty acid esters, transesterification products of oils and alcohols, polyglycerized fatty acids, sterol and sterol derivatives, PEG sorbitan fatty acid esters, PEG alkyl ethers, Sugar esters, PEG alkyl phenols, poloxamers, ionic surfactants, polyoxyethylene fatty acid esters, polysorbates, and reaction products of a natural or hydrogenated castor oil and ethylene oxide.

4. The composition of claim 1, wherein the super emulsifier comprises polysorbate 80 and sodium lauryl sulfate.

5. The composition of claim 4, wherein the polysorbate 80 and sodium lauryl sulfate is in a weight ratio of about 2:1 to about 3:1.

6. The composition of claim 1, wherein the binder is in an amount from about 0.01% to about 70% by weight.

7. The composition of claim 1, wherein the super emulsifier is in an amount from about 0.05% to about 15% by weight.

8. The composition of claim 1, wherein the diluent/bulking material is in an amount from about 10% to about 90% by weight.

9. The composition of claim 1, wherein the binder is povidone.

10. The composition of claim 1, further comprising a lubricant.

11. The composition of claim 10, wherein the lubricant is magnesium stearate, silicon dioxide, talc, or combinations thereof.

12. The composition of claim 1, wherein the emulsifier is adsorbed on a disintegrant.

13. The composition of claim 12, wherein the emulsifier is polysorbate 80 and the binder is crospovidone.

14. The composition of claim 1, wherein the diluent/bulking material is silicon dioxide, sugars, starches, lactose, sucrose, sorbitol, fructose, talc, stearic acid, magnesium stearate, dicalcium phosphate, erythitol, xylitol, mannitol, maltitol, isomalt, dextrose, maltose, lactose, microcrystalline celluloses, or mixtures thereof.

15. The composition of claim 1, wherein the composition dissolves in a mouth in less than about 45 seconds.

16. The composition of claim 1, wherein the composition is a flashbead or a compressed tablet.

17. A method for making rapid-melt compressed solid pharmaceutical composition, said method comprising the steps of
a. blending a binder and a super emulsifier to form a first mixture;
b. blending a diluent/bulking material and an active ingredient to form a second mixture;
c. blending the first and second mixtures; and
d. compressing the third mixture to form a tablet.

18. The method of claim 17, further comprising the step of compressing the mixture into a tablet using a compression force at about 18 to 32 KN.

19. The method of claim 17, wherein step c. further comprising the blending to lubricants with the first and second mixtures.

20. The method of claim 17, wherein the super emulsifier wherein the super emulsifier has a hydrophilic-lipophilic balance of greater than about 10.

21. The composition of claim 17, wherein the super emulsifier is selected from the group consisting of polyethoxylated fatty acids, polyethylene glycol (PEG) fatty acid diesters, PEG glycerol fatty acid esters, transesterification products of oils and alcohols, polyglycerized fatty acids, sterol and sterol derivatives, PEG sorbitan fatty acid esters, PEG alkyl ethers, Sugar esters, PEG alkyl phenols, poloxamers, ionic surfactants, polyoxyethylene fatty acid esters, polysorbates, and reaction products of a natural or hydrogenated castor oil and ethylene oxide.

22. A rapid-melt bead composition comprising:
a binder having a melting point from about 20 to about 90° C.;
an emulsifier;
a diluent/bulking material; and
an active ingredient.

23. The bead of claim 22, wherein the binder is cocoa butter.

24. The bead of claim 22, wherein the emulsifier is polysorbate 80, sodium lauryl sulfate, and/or PEG 3350.

25. The bead of claim 22, wherein the emulsifier is present at about 0.1-30% by weight of the composition.

26. A method for making a rapid-melt bead comprising the steps of
a. heating an emulsifier and a binder to a molten state to form a first mixture, wherein the emulsifier has a melting point from about 60 to about 40° C.;
b. mixing the active ingredient and diluent/bulking material to form a second mixture;
c. adding and mixing the first and second mixtures to form a third mixture;
d. extruding and spheronizing the third mixture to form beads.

27. The method of claim 26, further comprising the steps of drying the beads.

28. The method of claim 26, wherein step a. comprises heating to greater than about 180° F. and then maintaining the first mixture at about 115-140° F.

29. The method of claim 26, wherein step c. takes place at about 115-140° F.

30. The method of claim 26, wherein the wherein the binder is cocoa butter.

31. The method of claim 26, wherein the emulsifier is polysorbate 80, sodium lauryl sulfate, and/or PEG 3350.

32. A chew tablet composition comprising:
a binder;
an emulsifier having a melting point of greater than about 40° C.;
a diluent/bulking material; and
an active ingredient.

33. The chew tablet of claim 32, wherein the binder is at least one glyceride.

34. The chew tablet of claim 32, wherein the binder is monoglyceride, diglyceride, lecithin, or combinations thereof.

35. The chew tablet of claim 32, further comprising a lubricant, flavor, sweetener, or combinations thereof.

36. The chew tablet of claim 35, wherein the lubricant is magnesium stearate, silicon dioxide, talc, or combinations thereof.

37. The chew tablet of claim 32, wherein the emulsifier also acts as a binder.

38. The chew tablet of claim 32, wherein the active ingredient is present at about 0.1% (w/w) to 90% of the total chew tablet.

39. The chew tablet of claim 32, wherein the emulsifier is present at about 0.1-25% by weight of the composition.

40. A method for making a chew tablet comprising the steps of
a. heating an emulsifier and a binder to a molten state to form a first mixture, wherein the binder has a melting point of greater than about 40° C.;
b. mixing the active ingredient and diluent/bulking material to form a second mixture;
c. adding and mixing the first and second mixtures to form a third mixture and
d. compressing the third mixture to form a tablet.

41. The method of claim 40, wherein step a. comprises heating to greater than about 180° F. and then maintaining the first mixture at about 115-140° F.

42. The method of claim 40, wherein tableting occurs at a compression force at about 20 to about 25 KN.

43. The method of claim 40, wherein step b. also include the addition of lubricants, flavors, and sweeteners.

44. The method of claim 43, wherein the lubricant is magnesium stearate, silicon dioxide, talc, or combinations thereof.

45. The method of claim 40, wherein the wherein the binder is monoglyceride, diglyceride, lecithin, or combinations thereof.

46. The method of claim 40, wherein the emulsifier also acts as a binder.

47. The method of claim 40, wherein the active ingredient is present at greater than 40% (w/w) of the total chew tablet.

48. A composition comprising
first granules or beads having a binder having a melting point about 20-90° C., an emulsifier, a diluent/bulking material; and
a second granules or beads having an active ingredient.

49. The composition of claim 48, wherein the emulsifier is cocoa butter.

50. The composition of claim 48, wherein the emulsifier is polysorbate 80, sodium lauryl sulfate, and/or PEG 3350.

51. The composition of claim 48, wherein the emulsifier is present at about 0.1-25% by weight of the composition.

52. The composition of claim 48, wherein the active ingredient is coated.
